(19) United States
(12) Patent Application Publication
(10) Pub. No.: US 2022/0220564 A1
(43) Pub. Date: Jul. 14, 2022

(54) IMPROVED METHODS FOR THE EARLY DIAGNOSIS OF UTERINE LEIOMYOMAS AND LEIOMYOSARCOMAS

(71) Applicant: IGENOMIX, S.L., Valencia (ES)

(72) Inventors: Aymara MAS PERUCHO, Valencia (ES); Roberto ALONSO VALERO, Valencia (ES); Carlos SIMON VALLES, Valencia (ES)

(21) Appl. No.: 17/604,316

(22) PCT Filed: Apr. 17, 2020
(86) PCT No.: PCT/EP2020/060878

§ 371 Date: Oct. 15, 2021

(30) Foreign Application Priority Data
Apr. 17, 2019 (EP) 19382307.7
Apr. 29, 2019 (EP) 19382322.6

(51) Int. Cl. C12Q 1/6886; A61B 17/32

(52) U.S. Cl. CPC C12Q 1/6886; A61B 17/32002; A61B 2017/320024; C12Q 2600/156; C12Q 2600/158

(57) ABSTRACT

The present disclosure provides a method for differentiating myometrial tumors/uterine neoplasms such as LM, LMS and IMT. Further, the disclosure provides a method for treating a uterine leiomyoma in a subject, comprising: (a) performing a genotyping assay on a biological sample from the subject to determine whether the subject has a uterine leiomyosarcoma genotype, and (b) surgically removing the uterine leiomyoma if the subject does not have a uterine leiomyosarcoma genotype.

### FIELD OF THE INVENTION

The specification relates to improved methods for the early preoperative diagnosis of uterine leiomyomas and leiomyosarcomas to help prevent accidental malignant dissemination derived from surgical methods like morcellation.

### BACKGROUND OF THE INVENTION

Uterine leiomyomas (LM) are benign smooth muscle tumors with an estimated lifetime risk of ˜70% of women at reproductive age.¹ These tumors produce complications including pelvic pain, heavy menstrual bleeding, anemia, infertility, and recurrent pregnancy loss.^{2,3} Although selective progesterone receptor modulators are used to manage LM,⁴⁻⁵ surgery remains the long-term therapeutic option. Specifically, laparoscopic myomectomy with morcellation is the gold standard intervention,⁶ particularly for women who wish to preserve their fertility.⁷ However, this surgery carries potential detrimental effects for patients with undiagnosed occult leiomyosarcoma (LMS).⁸

LMS represents 70% of all uterine sarcomas, but remains rare, with an incidence of 0.4-0.9 in 100,000 women.⁹ They are aggressive malignant tumors, arising from the myometrium, characterized by early metastasis, poor prognosis, and high rates of recurrence with limited therapeutic efficacy.¹⁰⁻¹² The risk of occult uterine cancer in women with benign lesions is 1/350, and clinical symptoms as well as morphological features between LM and LMS are indistinguishable.¹³⁻¹⁸ Therefore, there is a risk of hidden malignancy during surgery.

Researchers have since attempted to develop preoperative diagnostic tests to discriminate between benign and malignant uterine masses.²² However, no clear evidence indicates that vaginal ultrasound and elastography,^{23,24} or magnetic resonance imaging and computed tomographyl^{11,25} can discriminate LM and LMS. Further, observations of a differential protein pattern are hampered by false-positive findings.²⁶

Lack of an accurate preoperative or intra-operative diagnostic to differentiate myometrial tumors affect their surgical treatment. FDA regulation has substituted laparoscopic myomectomy for laparotomy-based procedures, increasing morbidity, mortality, and cost for the patient and healthcare system.³⁴ The present specification discloses the existence of consistent differential genetic alterations at the genomic and transcriptomic levels between LMS and LM to establish an early differential diagnosis to improve treatment and management of LM.

### SUMMARY

The instant specification provides a system that allows clinicians to utilize genomic tools, genetic variants and possible transcriptomic and genomic markers in a new tool to effectuate the differential molecular diagnosis of myometrial tumors/uterine neoplasms such as LM (leiomyoma), LMS (leiomyosarcoma) and IMT (inflammatory myofibroblastic tumor) through an integrated comparative genomic and transcriptomic analysis. This provides a solution to a major problem in the current clinical approach to common uterine neoplasms by providing a tool that clinicians can use to evaluate the risk that apparently benign tumors are in fact rarer but much more dangerous malignant neoplasms.

Thus, in one aspect, the disclosure provides a method for diagnosing a myometrial tumor in a subject, comprising a unique integrative molecular analysis of transcriptomic and genomic data on a biological sample (e.g., tumoral tissue) from the subject, to determine whether the subject has a LM, LMS and/or IMT profile and/or genotype.

In another aspect, the disclosure provides a method for diagnosing a myometrial tumor in a subject, comprising performing a genotyping assay on a biological sample from the subject to determine whether the subject has a LM genotype. In various embodiments, the genotyping assay involved the detection of one or more biomarkers that are indicative of LM.

In still another aspect, the disclosure provides a method for diagnosing a myometrial tumor in a subject, comprising performing a genotyping assay on a biological sample from the subject to determine whether the subject has an LMS genotype. In various embodiments, the genotyping assay involved the detection of one or more biomarkers that are indicative of LMS.

In yet another aspect, the disclosure provides a method for diagnosing a myometrial tumor in a subject, comprising performing a genotyping assay on a biological sample from the subject to determine whether the subject has an IMT genotype. In various embodiments, the genotyping assay involved the detection of one or more biomarkers that are indicative of IMT. In other aspect, the methods for diagnosing LM, LMT, or IMT can be combined with a therapeutic method or treatment step for treating a myometrial tumors/uterine neoplasm (e.g., a leiomyoma or leiomyosarcoma).

Thus, in one aspect, the disclosure provides a method for treating a myometrial tumor in a subject, comprising: (a) performing a genotyping assay on a biological sample from the subject to determine whether the subject has a uterine leiomyosarcoma genotype, and (b) surgically removing the myometrial tumor if the subject does not have a uterine leiomyosarcoma genotype.

In various embodiments, the method further comprises performing a second genotyping assay on the biological sample to confirm whether the subject has a uterine leiomyoma genotype before step (b).

In other embodiments, the disclosure provides a method of treating a subject having a myometrial tumor , comprising performing a genotyping assay on a biological sample obtained from the subject, and removing the myometrial tumor from the subject, wherein the genotyping assay indicates that the subject does not have a uterine leiomyosarcoma (i.e., confirming that the tumor is benign and/or not malignant).

In still other embodiments, the disclosure provides biomarkers which are indicate that a myometrial tumor comprises a leiomyosarcoma.

In yet other embodiments, the disclosure provides biomarkers which are indicative that a myometrial tumor is a leiomyoma.

In various embodiments, the uterine leiomyosarcoma genotype (i.e., a malignant genotype of a myometrial tumor) comprises the detection of a mutation in one or more of the following biomarkers: FGF8, RET, PTEN, ATM, CADM1, KMT2A, NOTC H2, MCL1, DDR2, CCND1, FGF19, FGF3, MDM4, KRAS, SDCCAG8, CCND2, RP11-61102.2, MDM2, ARID1A, FGF14, LAMP1, NA, FG, F9, FLT1, ALOX5AP, BRCA2, RB1, MYCL, MPL, H PDL, MUTYH, RAD54L, RAD51B, FANCI, TSC2, P ALB2, NLRC3, SLX4, CREBBP, CDH1, RP11525K10.1, RAP1GAP2, RAD51L3-RFFL, ERBB2, BRCA1, TEX14, RPS6KB1, TP53, RBFOX3, BCL2, STK11, NOTCH3, JAK3, TGFBR 3, CCNE1, AKT2, ERCC2, PPP1R13L, PIK3CD, G NAS, ERG, ERBB4, BARD1, RNA5SP495, CHEK2, RP1-302D9.3, EP300, RNU6-688P, MSH6, VHL, MKRN2, ATR, MLH1. TET2, F GF2, FGFR3, PDGFRA, KDR, FGF5, APC, HMGX B3, CSF1R, PDGFRB, FGF10, PIK3R1, DHFR, RO S1, HIVEP1, ESR1, BYSL, MET, SMO, BRAF, DPP 6, CARD11, EGFR, CASC11, NRG1, FGFR1, NOT CH1, MLLT3, LINGO2, PTCH1, and AR (e.g., combinations of 2, 3, 4, 5, 6, 7, 8, 9, or 10 or more of the indicated biomarkers).

In various embodiments, the one or more mutations associated with a leiomyoma or leiomyosarcma genotype are deletions (DEL), insertions (INS), or a single-nucleotide polymorphisms (SNP).

In still other embodiments, the uterine leiomyoma genotype (i.e., a benign genotype of a myometrial tumor) comprises the detection of a mutation in one or more of the following biomarkers: FGFR2, KLLN, PTEN, ATM, KMT2A, MTOR, NRA S, NOTCH2, FGF19, AP001888.1, FGF3, MRE11 A, MDM4, PTPN11, SDCCAG8, FGF6, ERBB3, M DM2, NA, LAMP1, FGF9, FLT1, BRCA2, MYCL, RP 11-982M15.2, MPL, HPDL, SLC35F4, RAD51B, RAD 51, IDH2, TSC2, SLX4, CREBBP, RAD51L3-RFFL, TP53, RBFOX3, STK11, NOTCH3, TGFBR 3, AKT2, GNAS-AS1, ERG, MYCNOS, BARD1, EP300, DNMT3A, MSH2, MSH6, VHL, RAF1, PIK3CB, PIK3CA, TFR C, MLH1, BAP1, TET2, FGFR3, PDGFRA, MRPS1 8C, APC, HMGXB3, CSF1R, PDGFRB, FGFR4, F GF10, ESR1, BYSL, CCND3, SMO, DPP6, EGFR, CDK6, MYC, NRG1, NOTCH1, MLLT3, RP11-145E5.5, JAK2, GNAQ, PTCH1, and AR (e.g., combinations of 2, 3, 4, 5, 6, 7, 8, 9, or 10 or more of the indicated biomarkers).

In various other embodiments, the uterine leiomyosarcoma genotype comprises the detection of a mutation in one or more of the following biomarkers: FGF1, JAK2 KRAS, CDK4, FGF10, FGF5, MYC, FGF14, FGF7, MDM4, MYCL1, and NRG1 (e.g., combinations of 2, 3, 4, 5, 6, 7, 8, 9, or 10 or more of the indicated biomarkers).

In still other embodiments, the uterine leiomyoma genotype comprises the detection of a mutation in one or more of the following biomarkers: CCND, FGFR3, and MET

In still other embodiments, the uterine leiomyosarcoma genotype comprises the detection of a mutation in one or more of the following biomarkers: CDK4, FGF10, FGF5, MYC, MYCL1, NRG1, FGF1, FGF14, JAK2, KRAS, FGF14 FGF7, MDM4, MYCL1, NRG1, FGF5, RET, ALK, BRCA2, FGFR3, FGFR4, FLT3, NTRK1, PAX3, PAX7, RET, ROS1, and TMPRSS2 (e.g., combinations of 2, 3, 4, 5, 6, 7, 8, 9, or 10 or more of the indicated biomarkers).

In other embodiments, the uterine leiomyosarcoma genotype comprises the detection of a CNV duplication mutation in one or more of the following biomarkers: CDK4, FGF10, FGF5, MYC, MYCL1, and NRG1.

The uterine leiomyosarcoma genotype can also comprise the detection of a CNV deletion mutation in one or more of the following biomarkers: FGF1, FGF14, JAK2, and KRAS.

In other embodiments, the uterine leiomyosarcoma genotype comprises the detection of a CNV deletion & duplication mutation in one or more of the following biomarkers: FGF14, FGF7, MDM4, MYCL1, and NRG1.

In yet other embodiments, the uterine leiomyosarcoma genotype comprises the detection of an SNV mutation in one or more of the following biomarkers: FGF5 and RET.

In still other embodiments, the uterine leiomyosarcoma genotype comprises the detection of mRNA upregulation in ALK, BRCA2, FGFR3, FGFR4, FLT3, NTRK1, PAX3, PAX7, RET, ROS1, and TMPRSS2 (e.g., combinations of 2, 3, 4, 5, 6, 7, 8, 9, or 10 or more of the indicated biomarkers).

The uterine leiomyoma genotype can comprise in other embodiments the detection of a mutation in one or more of the following biomarkers: FGF3 and MET.

The uterine leiomyoma genotype can also comprise the detection of a CNV duplication mutation in FGFR3.

The uterine leiomyoma genotype can also comprise the detection of a CNV deletion mutation in MET.

The method of claim 1, wherein the uterine leiomyoma is a subserous fibroid, an intramural fibroid, or a submucous fibroid.

The uterine leiomyoma can be a submucous leiomyoma having a grade 0, grade 1, or grade 2 uterine leiomyoma.

In various embodiments, the methods involved analyzing or genotyping a myometrial tumor or a biological sample from a subject with a myometrial tumor, wherein the myometrial tumor comprises a leiomyoma (LM), a leiomyosarcoma (LMS), and/or an inflammatory myofibroblastic tumor (IMT).

The uterine leiomyoma can be a subserous fibroid, an intramural fibroid or a submucous fibroid.

The submucous uterine leiomyoma can be a grade 0, grade 1, or grade 2 uterine leiomyoma.

In various embodiments, the biological sample that is obtained can be a biological fluid, which can be, but is not limited to, blood, blood plasma, or urine. The biological sample can also be a biological tissue, such as a myometrial tumor (or biospy thereof).

The DNA sample can also be genomic DNA from the myometrial tissue and/or a cell-free tumor DNA (cftDNA) sample, e.g., from a blood or blood plasma sample.

In various embodiments, the genotyping assay can be a restriction fragment length polymorphism identification (RFLPI) of the DNA sample, a random amplified polymorphic detection (RAPD) of the DNA sample, an amplified fragment length polymorphism (AFLPD) of the DNA sample, a polymerase chain reaction (PCR) of the DNA sample, DNA sequencing of the DNA sample, or hybridization of the DNA sample to a nucleic acid microarray.

The DNA sequencing can be a next-generation sequencing method, such as single-molecule real-time sequencing (SMRT), ion semiconductor sequencing, pyrosequencing, sequencing by synthesis, combinatorial probe anchor synthesis (cPAS), sequencing by ligation (SOLiD sequencing), nanopore sequencing, or massively parallel signature sequencing (MPSS).

In various embodiments, the step of surgical removal of the uterine leiomyoma is by laparoscopic morcellation or myomectomy.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIGS. 1A-1D show the comparative genomic analysis of leiomyoma (LM) and leiomyosarcoma (LMS). FIG. 1A depict pie charts showing percentage of copy number variations (CNV) in LM and LMS (top to bottom). FIG. 1B shows a profile for detected amplifications and deletions in LM (top) and LMS samples (bottom) using log²-fold change (FC). FIG. 1C depicts barplots showing distribution of deletions and duplications per sample (left). Barplots showing distribution of deletions and duplications associated by gene (right). FIG. 1D is a Venn diagram representing number of shared CNVs by uterine LM (right) and LMS (left).

FIGS. 2A-2B show the clustering of LM, LMS and IMT samples based on CNV FIG. 2A shows principal component analysis (PCA) of leiomyoma (LM) (N=13), leiomyosarcoma (LMS) (N=13) and IMT (N=1) samples. Each sample is represented in the figure as a colored point (green, LMS; purple, LM; yellow, IMT). Most variance between both groups is captured in the first two principal components. FIG. 2B is a heatmap dendrogram of CNVs associated with genes (column) and for each analyzed sample (row) of LM (purple), LMS (green) and IMT (yellow). Copy number profiles including frequent amplifications (red) and deletions (blue). Horizontal length of each arm reflects relatedness of clusters.

FIGS. 3A-3C show targeted transcriptional profile for the 55 genes included in the TruSeq Tumor 170 gene panel. FIG. 3A is a multidimensional scaling plot of distances (MDS) in leiomyoma (LM) (N=13), leiomyosarcoma (LMS) (N=13) and IMT (N=1) samples in gene expression profiles. Each sample is represented in the figure as a colored point (green, LMS; purple, LM; yellow, IMT). Most variance between both groups is captured in the first two principal components. FIG. 3B is a heatmap dendrogram of expression of the 55 genes analyzed (column) for each sample (row), showing three clusters of samples. FIG. 3C is a boxplot for 11 genes significantly upregulated in LMS (green) vs. LM (purple). Thep-value is represented for each gene.

FIGS. 4A-4C show the detection of a novel ALK-TNS1 fusion transcript in IMT specimen, initially diagnosed as LMS. FIG. 4A is a schematic representation of the gene sequence and main functional domains of proteins for TNS1 and ALK. In the gene sequence, red arrow indicates the exon where the fusion was detected. In the protein scheme, black lines represent breakpoints, and dashed lines indicate closer view of the transcript fusion point. Amino acid sequence at the fusion point is highlighted in rectangle. FIG. 4B shows immunohistochemistry staining of intense cytoplasmic staining for ALK in the IMT01 sample. Scale bar represents 200 μM. FIG. 4C is a representative image of fluorescence in situ hybridization (FISH) for ALK showing several nuclei harboring split and fused signals (arrows).

FIG. 5 shows an integrative representation of recurrently affected genes in leiomyosarcomas. Columns represent samples, as indicated at the bottom, while most representative genes are shown by rows. Grey boxes indicate unaffected genes. Blue boxes indicate genes affected with deletions, and red boxes denote duplications. Mutations are represented by black squares, while highlighted green squares indicate mRNA upregulation.

FIGS. 6A-6F show the functional meaning of integrated signature for the tumorigenic process. FIG. 6A shows the distribution of implicated functions based on KEGG pathway database, where pathways, classified based on p-adjust value, are represented on the y-axis and number of genes belonging to each pathway are detailed on the x-axis. FIG. 6B shows the PI3K-AKT signaling pathway diagram containing fold-change representation for most integrated genes belonging to this pathway. FIG. 6C shows functional gene annotation in KEGG for specific molecular functions based on p-adjust value. FIG. 6D shows network modeling of gene expression and functional relationship between all specific processes related to molecular functions. Big nodes represent main categorical functions in the related process, while small spheres represent genes obtained by integration analysis. FIG. 6E shows the functional gene annotation in KEGG for specific biological processes based on p-adjust value. FIG. 6F shows the network modeling of gene expression and functional relationship between all specific biological processes.

FIGS. 7A-7C: FIG. 7A shows the distribution of implicated functions based on KEGG pathway database, where pathways are represented on the y-axis and number of genes belonging to each pathway are detailed on the x-axis. FIG. 7B shows GO enrichment analysis of molecular functions containing pathway name and gene ratio from the annotated signature. FIG. 7C shows GO enrichment analysis of biological process. The p-adjust value representation was showed as a gradient color from blue to red.

### DETAILED DESCRIPTION

The present disclosure describes an innovative tool that allows clinicians to utilize genomic tools, genetic variants and possible transcriptomic and genomic markers in a new tool to effectuate the differential molecular diagnosis of myometrial tumors/uterine neoplasms such as LM, LMS and IMT. This provides a solution to a major problem in the current clinical approach to common uterine neoplasms by providing a tool that clinicians can use to evaluate the risk that apparently benign tumors are in fact rarer but much more dangerous malignant neoplasms. Based on the databases developed by the inventors, it is proposed that a diagnostic tool driven principally by “Next Generation Sequencing” of DNA and RNA originating in the neoplastic tissue differentiates uterine LMS and LM is a manner that cannot be achieved by histological techniques or any other current diagnostic method.

### Definitions

Unless defined otherwise, all technical and scientific terms used herein have the meaning commonly understood by one of ordinary skill in the art to which this invention belongs. The following references provide one of skill in the art to which this invention pertains with a general definition of many of the terms used in this invention: Singleton et al., Dictionary of Microbiology and Molecular Biology (2d ed. 1994); The Cambridge Dictionary of Science and Technology (Walker ed., 1988); Hale & Marham, The Harper Collins Dictionary of Biology (1991); and Lackie et al., The Dictionary of Cell & Molecular Biology (3d ed. 1999); and Cellular and Molecular Immunology, Eds. Abbas, Lichtman and Pober, 2nd Edition, W. B. Saunders Company. For the purposes of the present invention, the following terms are further defined.

As used herein and in the claims, the singular forms “a,” “an,” and “the” include the singular and the plural reference unless the context clearly indicates otherwise. Thus, for example, a reference to “an agent” includes a single agent and a plurality of such agents.

The term “subject” or “patient” refers herein to a person in need of the analysis described herein. In some embodiments, the subject is a patient. In some embodiments, the subject is a human. In some embodiments, the subject is a female human (a woman). In some embodiments the subject is a female presenting with pathology and or history consistent with uterine fibroids believed to be a benign neoplasm. In some embodiments the subject is a female presenting with pathology and or history consistent with uterine fibroids believed to be leiomyoma (LM). In some embodiments the subject is a female presenting with pathology and or history consistent with uterine fibroids believed to be leiomyoma and desiring surgical intervention. In some embodiments the subject is a female presenting with pathology and or history consistent with uterine fibroids believed to be leiomyoma, desiring surgical intervention and requiring an evaluation of the neoplasm to evaluate the risk that the neoplasm is malignant in order to guide the selection of therapy. In some embodiments the subject is a female presenting with pathology and or history consistent with uterine fibroids believed to be leiomyoma, desiring surgical intervention and requiring an evaluation of the neoplasm to evaluate the risk that the neoplasm is a sarcoma in order to guide the selection of therapy. consistent with uterine fibroids believed to be leiomyoma and desiring surgical intervention. In some embodiments the subject is a female presenting with pathology and or history consistent with uterine fibroids believed to be leiomyoma (LM), desiring surgical intervention and requiring an evaluation of the neoplasm to evaluate the risk that the neoplasm is leiomyosarcoma in order to guide the selection of therapy.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

The term “genotype” as used herein refers to the genetic information an individual carries at one or more positions in the genome. A genotype may refer to the information present at a single polymorphism, for example, a single SNP. For example, if a SNP is bi-allelic and can be either an A or a C then if an individual is homozygous for A at that position the genotype of the SNP is homozygous A or AA. Genotype may also refer to the information present at a plurality of polymorphic positions. A genotype may also refer to other genetic signatures or mutations, such as insertions or deletions in a gene, or to one more more duplicated or repeated portions of a gene, or to inversions, or to frameshift mutations, and the like. A genotype may also include epigenetic genotypes, i.e., wherein the biomarker is an altered pattern of methylation in a gene.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3rd Ed., W. H. Freeman Pub., New York, N.Y. and Berg et al. (2002) Biochemistry, 5th Ed., W. H. Freeman Pub., New York, N.Y., all of which are herein incorporated in their entirety by reference for all purposes.

### Biological Samples

Any suitable biological sample may be used in the present methods to evaluate and detect a LM or a LMS.

In an embodiment, the biological sample is blood.

In another embodiment, the biological sample is plasma.

In still another embodiment, the biological sample is from a bodily tissue or organ. The bodily tissue or organ can include uterus, brain, connective, bone, muscle, nervous system, lymph system, lungs, heart, blood vessels, stomach, colon, small intestine, pancreas, or gall bladder. Preferably, the sample is from a subject having or having a LM or a LMS.

In some embodiments, a biological sample is obtained when a subject develops one or more signs or symptoms that are characteristic of LM or LMS.

In some embodiments, a biological sample is obtained after subject has had one or more signs or symptoms of LM or LMS at least several days (for example 2-5 days, 5-10 days, 1-2 weeks, 2-4 weeks, or longer). In other embodiments, the subject does not need to have signs or symptoms in advance of a diagnosis using the herein described methods.

As described herein, when a reference is made to obtaining or evaluating a biological sample it should be understood that one or more biological samples (e.g., two, three, four, five, six, seven, eight, nine, ten, or more biological samples) may be obtained or evaluated (e.g., for each subject). In addition, in certain embodiments, samples may be taken over a period of time and evaluated to determine a condition over time, e.g., several day to several months to several years.

In some embodiments, a biological sample may be a blood sample. In some embodiments, a biological sample may be a non-blood sample.

In some embodiments, a sample may be processed to remove cells in order to produce a cell-free sample (e.g., cell-free plasma or serum). In some embodiments, cells may be removed from a sample via centrifugation, chromatography, electrophoresis, or any other suitable method.

The biological samples may be used directly as obtained from the biological source or following a pretreatment to modify the character of the sample. For example, such pretreatment may include preparing plasma from blood, diluting viscous fluids and so forth. Methods of pretreatment may also involve, but are not limited to, filtration, precipitation, dilution, distillation, mixing, centrifugation, freezing, lyophilization, concentration, amplification, nucleic acid fragmentation, inactivation of interfering components, the addition of reagents, lysing, etc. If such methods of pretreatment are employed with respect to the sample, such pretreatment methods are typically such that the nucleic acid(s) of interest remain in the test sample, preferably at a concentration proportional to that in an untreated test sample (e.g., namely, a sample that is not subjected to any such pretreatment method(s)). Such “treated” or “processed” samples are still considered to be biological “test” samples with respect to the methods described herein.

In some embodiments, the sample is a mixture of two or more biological samples, e.g., a biological sample can comprise two or more of a biological fluid sample, a tissue sample, and a cell culture sample. As used herein, the terms “blood,” “plasma” and “serum” expressly encompass fractions or processed portions thereof. Similarly, where a sample is taken from a biopsy, swab, smear, etc., the “sample” expressly encompasses a processed fraction or portion derived from the biopsy, swab, smear, etc.

In various embodiments, the biological sample (e.g., blood or plasma) is treated or processed by known methods to obtain the cell-free DNA present therein.

In the present invention, analysis of the genotype, genetic signature or biomarker signature, or additionally the expression signature or transcriptomic signature may be undertaken on any biologic sample, comprising tissue, isolated cells, or biological fluid comprising nucleic acids derived from the patient's uterine neoplasm. For the purposes of this disclosure, nucleic acids comprise DNA and RNA (e.g., genomic DNA and messenger RNA, respectively). For the purposes of this invention tissue comprises a multicellular portion of the neoplasm obtained by surgical resection or biopsy of a confirmed or suspected uterine neoplasm. Isolated cells may be obtained by biopsy of the suspected or confirmed neoplasm, for example, by needle biopsy, transcervical endometrial biopsy, or dilation and curettage (also known as D&C). Isolated cells may also be obtained by sampling of the myometium, for example, by obtaining a biospy of the respective tissue to recover cellular material including material shed from the neoplasm. A biological fluid comprising nucleic acids may also be used to sample and detect nucleic acids originating in the uterine neoplasm subject to further bioinformatic analyses known in the art as methods of determining the tissue of origin for such cell free nucleic acids. Such biological fluids comprise whole blood, serum, plasma, lymph fluid, urine, mucus, saliva or myometrium biopsies. In certain embodiments, the biological sample is a fluid, such as blood or blood plasma.

Nucleic acids may be extracted from the biological samples using methods known in the art such as extraction to a solid phase resin or bead or phenol-chloroform extraction or other organic extraction, with DNA specific degrading enzymatic treatments and RNA degrading enzyme inhibitors to enrich for RNA as required. Where necessary such methods can include techniques known in the art to be useful for the recovery of nucleic acids from formalin-fixed paraffin embedded tissue in order to enable the method of the present invention to be practiced on histology samples previously obtained from the patient without the need to obtain an additional biological sample.

### Biomarkers

As used herein, the term “biomarker” or “biological marker” refers to a broad subcategory of medical signs—that is, objective indications of medical state observed from outside the patient—which can be measured accurately and reproducibly. One or more such biomarkers may be present in a specific population of cells (e.g., cells obtained in biopsy of tissue that is visually identified as neoplastic or alternate, histologically confirmed by microscopic examination with or without stains to represent tissue with neoplastic differentiation with respect to the surrounding tissue) and the level of each biomarker may deviate from the level of the same biomarker in a different population of cells and/or in a different subject (e.g., patient). For example, a biomarker that is indicative of leiomyosarcoma may have an elevated level or a reduced level in a sample from a subject (e.g., a sample from a subject that has or is at risk for leiomyosarcoma) relative to the level of the same marker in a control sample (e.g., a sample from a normal subject, such as a subject who does not have or is not at risk for leiomyosarcoma).

Combined groups of biomarkers with a uniquely characteristic pattern associated with a condition, disease, or otherwise biological state (e.g., leiomyoma or leiomyosarcoma) may be referred to as a “biomarker signature” or equivalently as a “gene signature” or “gene expression signature” or “gene expression profile.” A gene signature or gene expression signature is a single or combined group of genes in a cell with a uniquely characteristic pattern of gene expression that occurs as a result of a biological process or pathogenic medical condition (e.g., leiomyoma or leiomyosarcoma). Activating pathways in a regular physiological process or a physiological response to a stimulus results in a cascade of signal transduction and interactions that elicit altered levels of gene expression, which is classified as the gene signature of that physiological process or response.

The clinical applications of gene signatures breakdown into prognostic, diagnostic, and predictive signatures. The phenotypes that may theoretically be defined by a gene expression signature range from those that predict the survival or prognosis of an individual with a disease, those that are used to differentiate between different subtypes of a disease (e.g., leiomyoma and leiomyosarcoma), to those that predict activation of a particular pathway. Ideally, gene signatures can be used to select a group of patients for whom a particular treatment will be effective (e.g., medical treatment or minimally invasive surgical procedures for confirmed LM versus more invasive, urgent and multifactorial treatment modalities appropriate to LMS).

Prognostic refers to predicting the likely outcome or course of a disease. Classifying a biological phenotype or medical condition based on a specific gene signature or multiple gene signatures, can serve as a prognostic biomarker for the associated phenotype or condition (e.g., leiomyoma or leiomyosarcoma). This concept termed prognostic gene signature, serves to offer insight into the overall outcome of the condition regardless of therapeutic intervention. Several studies have been conducted with focus on identifying prognostic gene signatures with the hopes of improving the diagnostic methods and therapeutic courses adopted in a clinical setting. It should be noted that prognostic gene signatures are not themselves a target of therapy but they offer additional information to consider when planning a therapeutic intervention. The criteria a gene signature preferably meets to be deemed a prognostic marker include demonstration of its association with the outcomes of the condition, reproducibility and validation of its association in an independent group of patients and lastly, the prognostic value must demonstrate independence from other standard factors in a multivariate analysis. In the instant invention prognostic signature is principally concerned with distinguishing those patients who are likely to have no recurrence or metastases as a result of the standard of care morcellation-based therapy indicated for canonical LM and similarly benign tumors versus those subjects who would be at elevated risk of recurrent and/or metastatic disease as sequelae to the same intervention due to the consequent dispersal of malignant cells from their canonical LMS or similarly malignant tumors.

A diagnostic gene signature serves as a biomarker that distinguishes phenotypically similar medical conditions that have a threshold of risk comprising risk acceptable for a given therapeutic intervention and risk unacceptable for the given therapeutic intervention. Establishing verified methods of diagnosing clinically indolent and malignant cases allows practitioners to provide risk adjusted therapeutic options that range from no therapy, additional diagnostic process for cases where the biomarkers indicated intermediate risk level, standard of care surgical intervention for acceptable risk cases to more aggressive surgical intervention potentially coupled with other therapeutic modalities such as immunotherapy, radiotherapy and/or chemotherapy in cases where there biomarker informed risk profile renders standard of care intervention unacceptably risky. Such diagnostic signatures also allow for a more accurate representation of test samples used in research.

A predictive gene signature predicts the effect of treatment in patients or study participants that exhibit a particular disease phenotype. A predictive gene signature unlike a prognostic gene signature can be a target for therapy. The information predictive signatures provide are more rigorous than that of prognostic signatures as they are based on treatment groups with therapeutic intervention on the likely benefit from treatment, completely independent of prognosis. Predictive gene signatures address the paramount need for ways to personalize and tailor therapeutic intervention in diseases. These signatures have implications in facilitating personalized medicine through identification of more novel therapeutic targets and identifying the most qualified subjects for optimal benefit of specific treatments, comprising surgical intervention other than laparoscopic power morcellation, for example en bloc tissue removal, for example, through the vagina or via a mini-laparotomy incision, or manual morcellation with or without tissue containment, any of which might be conducted with adjunctive antineoplastic therapy in higher risk cases.

Exemplary biomarkers indicative of leiomyoma and leiomyosarcoma are provided in, but not limited to, Tables 3, 6, 7, 8, and 9. In some embodiments, a biomarker signature (i.e., combinations of two or more biomarkers) may be constructed using combinations of biomarkers from Tables 3, 6, 7, 8 and/or 9. For example, one or more biomarkers from Table 3 may be combined with one or more biomarkers from Tables 6, 7, 8, and/or 9. In another embodiments, one or more biomarkers from Table 6 may be combined with one or more biomarkers from Tables 3, 7, or 8. In still other embodiments, one or more biomarkers from Table 7 may be combined with one or more biomarkers from Tables 3, 6, 8, and/or 9. In other embodiments, one or more biomarkers from Table 8 may be combined with one or more biomarkers from Tables 3, 6, 7, and/or 9. In still other embodiments, any first biomarker disclosed herein in any table or listing or set may be combined with any second biomarker disclosed. Further, this combination may be combined with any third, or fourth, or fifth, or sixth, or seventh, or eighth, or ninth, or tenth or more biomarkers disclosed anywhere herein. Such combinations of biomarkers for the detection of LM and/or LMS may be referred to biomarker signatures.

In some embodiments, a biomarker is differentially expressed in a sample from a subject that has a malignant uterine tumor compared to a sample from a subject that does not have a malignant uterine tumor, or a subject that has benign uterine neoplasms. In some embodiments, a biomarker is differentially expressed in a sample from a subject that has leiomyosarcoma compared to a sample from a subject that does not have leiomyosarcoma, or a subject that has leiomyoma. In some embodiments, a biomarker is differentially expressed in a sample from a subject that has leiomyoma compared to a sample from a subject that does not have leiomyoma, or a subject that has leiomyosarcoma.

In some embodiments, the biomarker signature indicative of leiomyosarcoma (LMS) comprises one or more biomarkers (e.g., combinations of any 2, 3, 4, 5, 6, 7, 8, 9, 10 or more biomarkers) selected from the group consisting of FGF8, RET, PTEN, ATM, CADM1, KMT2A, NOTCH2, MCL1, DDR2, CCND1, FGF19, FGF3, MDM4, KRAS, SDCCAG8, CCND2, RP11-611O2.2, MDM2, ARID1A, FGF14, LAMP1, NA, FGF9, FLT1, ALOX5AP, BRCA2, RB1, MYCL, MPL, HPDL, MUTYH, RAD54L, RAD51B, FANCI, TSC2, PALB2, NLRC3, SLX4, CREBBP, CDH1, RP11-525K10.1, RAP1GAP2, RAD51L3-RFFL, ERBB2, BRCA1, TEX14, RPS6KB1, TP53, RBFOX3, BCL2, STK11, NOTCH3, JAK3, TGFBR3, CCNE1, AKT2, ERCC2, PPP1R13L, PIK3CD, GNAS, ERG, ERBB4, BARD1, RNA5SP495, CHEK2, RP1-302D9.3, EP300, RNU6-688P, MSH6, VHL, MKRN2, ATR, MLH1, TET2, FGF2, FGFR3, PDGFRA, KDR, FGF5, APC, HMGX B3, CSF1R, PDGFRB, FGF10, PIK3R1, DHFR, RO S1, HIVEP1, ESR1, BYSL, MET, SMO, BRAF, DPP 6, CARD11, EGFR, CASC11, NRG1, FGFR1, NOTCH1, MLLT3, LINGO2, PTCH1, and AR.

In some embodiments, the biomarker signature indicative of leiomyosarcoma (LMS) comprises a copy number variant (CNV) duplication in one or more biomarkers selected from the group consisting of CDK4, FGF10, FGF5 and MYC. In some embodiments, the biomarker signature indicative of leiomyosarcoma (LMS) comprises a copy number variant (CNV) duplication in CDK4, FGF10, FGF5 and MYC. In some embodiments, the gene signature indicative of leiomyosarcoma comprises a CNV deletion in one or more biomarkers selected from the group consisting of FGF1, JAK2, and KRAS. In some embodiments, the gene signature indicative of leiomyosarcoma comprises a CNV deletion in FGF1, JAK2, and KRAS. In some embodiments, the biomarker signature indicative of leiomyosarcoma comprises a CNV duplication and deletion in one or more biomarkers selected from the group consisting of FGF14, FGF7, MDM4, MYCL1, and NRG1. In some embodiments, the biomarker signature indicative of leiomyosarcoma comprises a CNV duplication and deletion in FGF14, FGF7, MDM4, MYCL1, and NRG1.

In some embodiments, the biomarker signature indicative of leiomyosarcoma (LMS) comprises one or more, or two or more, or three or more, or four or more, or five or more, or six or more, or seven or more, or eight or more, or nine or more, or ten or more, or eleven or more, or twelve or more, or thirteen or more, or fourteen or more, or fifteen or more, or sixteen or more, or seventeen or more, or eighteen or more, or nineteen or more, or twenty or more, or twenty-one or more, or twenty-two or more, or twenty-three or more, or twenty-four or more, or up to all of the biomarkers selected from the group consisting of (1) CDK4, (2) FGF10, (3) FGF5, (4) MYC, (5) MYCL1, (6) NRG1, (7) FGF1, (8) FGF14, (9) JAK2, (10) KRAS, (11) FGF7, (12) MDM4, (13) FGF5, (14) RET, (15) ALK, (16) BRCA2, (17) FGFR3, (18) FGFR4, (19) FLT3, (20) NTRK1, (21) PAX3, (22) PAX7, (23) RET, (24) ROS1, and (25) TMPRSS2. In various embodiments, the biomarker signature indicative of leiomyosarcoma comprises any combination of 2, or 3, or 4, or 5, or 6, or 7, or 8, or 9, or 10, or 11, or 12, or 13, or 14, or 15, or 16, or 17, or 18, or 19, or 20, or 21, or 22, or 23, or 24, or 25 or the biomarkers selected from the group consisting of: (1) CDK4, (2) FGF10, (3) FGF5, (4) MYC, (5) MYCL1, (6) NRG1, (7) FGF1, (8) FGF14, (9) JAK2, (10) KRAS, (11) FGF7, (12) MDM4, (13) FGF5, (14) RET, (15) ALK, (16) BRCA2, (17) FGFR3, (18) FGFR4, (19) FLT3, (20) NTRK1, (21) PAX3, (22) PAX7, (23) RET, (24) ROS1, and (25) TMPRSS2. In some embodiments, the biomarker signature indicative of leiomyosarcoma (LMS) comprises CDK4, FGF10, FGF5, MYC, MYCL1, NRG1, FGF1, FGF14, JAK2, KRAS, FGF7, MDM4, FGF5, RET, ALK, BRCA2, FGFR3, FGFR4, FLT3, NTRK1, PAX3, PAX7, RET, ROS1, and TMPRSS2. In some embodiments, the biomarker signature indicative of leiomyosarcoma comprises one or more, or at least 2, 3, 4, 5, 6, 7, 8, 9, 10, or 11 biomarkers selected from the group consisting of ALK, BRCA2, FGFR3, FGFR4, FLT3, NTRK1, PAX3, PAX7, RET, ROS1, and TMPRSS2. In some embodiments, the biomarker signature indicative of leiomyosarcoma comprises ALK, BRCA2, FGFR3, FGFR4, FLT3, NTRK1, PAX3, PAX7, RET, ROS1, and TMPRSS2. In some embodiments, the biomarker signature indicative of leiomyosarcoma comprises upregulation of one or more, or at least 2, 3, 4, 5, 6, 7, 8, 9, 10, or 11 of the biomarkers selected from the group consisting of ALK, BRCA2, FGFR3, FGFR4, FLT3, NTRK1, PAX3, PAX7, RET, ROS1, and TMPRSS2. In some embodiments, the biomarker signature indicative of leiomyosarcoma comprises upregulation of ALK, BRCA2, FGFR3, FGFR4, FLT3, NTRK1, PAX3, PAX7, RET, ROS1, and TMPRSS2.

In some embodiments, the biomarker signature indicative of leiomyosarcoma (LMS) comprises one or more biomarkers (e.g., any combination of 2, 3, 4, 5, 6, 7, 8, 9, 10, 11, 12, 13, 14, 15, 16, 17, 18, or 19 biomarkers) selected from the group consisting of ALK, BARD1, BRCA2, CCNE1, CDK4, FGF1, FGF10, FGF5, FGFR3, FLT3, JAK2, KRAS, NTRK1, PAX3, PAX7, PTEN, RET, ROS1, and TMPRSS2. In some embodiments, the biomarker signature indicative of leiomyosarcoma (LMS) comprises ALK, BARD1, BRCA2, CCNE1, CDK4, FGF1, FGF10, FGFS, FGFR3, FLT3, JAK2, KRAS, NTRK1, PAX3, PAX7, PTEN, RET, ROS1, and TMPRSS2. In some embodiments, the biomarker signature indicative of leiomyosarcoma comprises mRNA upregulation in one or more biomarkers (e.g., any combination of 2, 3, 4, 5, 6, 7, 8, 9, 10, or 11 biomarkers) selected from the group consisting of ALK, BRCA2, FGFR3, FGFR4, FLT3, NTRK1, PAX3, PAX7, RET, ROS1, and TMPRSS2. In some embodiments, the biomarker signature indicative of leiomyosarcoma comprises mRNA upregulation in ALK, BRCA2, FGFR3, FGFR4, FLT3, NTRK1, PAX3, PAX7, RET, ROS1, and TMPRSS2.

In some embodiments, the biomarker signature indicative of leiomyosarcoma comprises deletion (partial or complete) of one or more biomarkers (e.g., any combination of 1, 2, 3, or 4 biomarkers) selected from the group consisting of FGF1, JAK2, KRAS, and PTEN. In some embodiments, the biomarker signature indicative of leiomyosarcoma comprises deletion (partial or complete) of FGF1, JAK2, KRAS, and PTEN. In some embodiments, the biomarker signature indicative of leiomyosarcoma comprises duplication of one or more biomarkers (e.g., any combination of 1, 2, or 3 biomarkers) selected from the group consisting of CDK4, FGF10, and FGF5. In some embodiments, the biomarker signature indicative of leiomyosarcoma comprises duplication of CDK4, FGF10, and FGFS. In some embodiments, the biomarker signature indicative of leiomyosarcoma comprises a mutation in one or more biomarkers (e.g., any combination of 1, 2, 3, or 4 biomarkers) selected from the group consisting of BARD1, CCNE1, FGF5, and RET. In some embodiments, the mutation is a single nucleotide polymorphism (SNP). In some embodiments, the biomarker signature indicative of leiomyosarcoma comprises a mutation in BARD1, CCNE1, FGF5, and RET. In some embodiments, the mutation is a single nucleotide polymorphism (SNP).

In some embodiments, the biomarker signature indicative of leiomyosarcoma (LMS) comprises a copy number variant (CNV) duplication in one or more biomarkers (e.g., combinations of any 2, 3, 4, 5, or 6 biomarkers) selected from the group consisting of CDK4, FGF10, FGF5, MYC, MYCL1, and NRG1. In some embodiments, the biomarker signature indicative of leiomyosarcoma (LMS) comprises a copy number variant (CNV) duplication in CDK4, FGF10, FGF5, MYC, MYCL1, and NRG1. In some embodiments, the gene signature indicative of leiomyosarcoma comprises a CNV deletion in one or more biomarkers (e.g., combinations of any 2, 3, or 4 biomarkers) selected from the group consisting of FGF1, FGF14, JAK2, and KRAS. In some embodiments, the gene signature indicative of leiomyosarcoma comprises a CNV deletion in FGF1, FGF14, JAK2, and KRAS. In some embodiments, the biomarker signature indicative of leiomyosarcoma comprises a CNV duplication and deletion in one or more biomarkers (e.g., combinations of any 2, 3, 4, or 5 biomarkers) selected from the group consisting of FGF14, FGF7, MDM4, MYCL1, and NRG1. In some embodiments, the biomarker signature indicative of leiomyosarcoma comprises a CNV duplication and deletion in FGF14, FGF7, MDM4, MYCL1, and NRG1.

In some embodiments, the biomarker signature indicative of leiomyosarcoma (LMS) comprises a single nucleotide variant (SNV) in one or more biomarkers selected from the group consisting of FGF5 and RET. In some embodiments, the biomarker signature indicative of leiomyosarcoma (LMS) comprises a single nucleotide variant (SNV) in FGF5 and RET. In some embodiments, the gene signature indicative of leiomyosarcoma comprises mRNA upregulation in one or more biomarkers (e.g., combinations of any 2, 3, 4, 5, 6, 7, 8, 9, 10, or 11 biomarkers) selected from the group consisting of ALK, BRCA2, FGFR3, FGFR4, FLT3, NTRK1, PAX3, PAX7, RET, ROS1, and TMPRSS2. In some embodiments, the gene signature indicative of leiomyosarcoma comprises mRNA upregulation in ALK, BRCA2, FGFR3, FGFR4, FLT3, NTRK1, PAX3, PAX7, RET, ROS1, and TMPRSS2.

In some embodiments, the biomarker signature indicative of leiomyoma (LM) comprises one or more biomarkers (e.g., combinations of any 2, 3, 4, 5, 6, 7, 8, 9, 10 or more biomarkers) selected from the group consisting of FGFR2, KLLN, PTEN, ATM, KMT2A, MTOR, NRAS, NOTCH2, FGF19, AP001888.1, FGF3, MRE11A, MDM4, PTPN11, SDCCAG8, FGF6, ERBB3, MDM2, NA, LAMP1, FGF9, FLT1, BRCA2, MYCL, RP11-982M15.2, MPL, HPDL, SLC35F4, RAD51B, RAD51, IDH2, TSC2, SLX4, CREBBP, RAD51L3-RFFL, TP53, RBFOX3, STK11, NOTCH3, TGFBR 3, AKT2, GNAS-AS1, ERG, MYCNOS, BARD1, EP300, DNMT3A, MSH2, MSH6, VHL, RAF1, PIK3CB, PIK3CA, TFRC, MLH1, BAP1, TET2, FGFR3, PDGFRA, MRPS18C, APC, HMGXB3, CSF1R, PDGFRB, FGFR4, FGF10, ESR1, BYSL, CCND3, SMO, DPP6, EGFR, CDK6, MYC, NRG1, NOTCH1, MLLT3, RP11-145E5.5, JAK2, GNAQ, PTCH1, and AR.

In some embodiments, the biomarker signature indicative of leiomyoma (LM) comprises one or more biomarkers selected from the group consisting of CCND1, FGFR3, and MET. In some embodiments, the biomarker signature indicative of leiomyoma (LM) comprises CCND1, FGFR3, and MET. In some embodiments, the biomarker signature indicative of leiomyoma comprises a CNV duplication in FGFR3. In some embodiments, the biomarker signature indicative of leiomyoma comprises a CNV deletion in MET. In some embodiments, the biomarker signature indicative of leiomyoma comprises a CNV duplication in CCND1 and FGFR3, and a CNV deletion in MET.

In some embodiments, the biomarker signature indicative of leiomyoma (LM) comprises a T-G mutation in chr12-4551244. In some embodiments, the gene signature indicative of leiomyoma comprises a G-T mutation in chr11-94192599. In some embodiments, the biomarker signature indicative of leiomyoma (LM) comprises a T-G mutation in chr12-4551244 and a G-T mutation in chr11-94192599.

In some embodiments, the biomarker signature indicative of leiomyosarcoma (LMS) comprises a C-A mutation in chr10-43597827. In some embodiments, the gene signature indicative of leiomyosarcoma comprises a TAA-T mutation in chr4-81206898. In some embodiments, the biomarker signature indicative of leiomyosarcoma (LMS) comprises a C-A mutation in chr10-43597827 and a TAA-T mutation in chr4-81206898.

This Application may reference the “status” or “state” of a biomarker in a sample. In various embodiments, reference to the “abnormal status or state” of a biomarker means the biomarker's status in a particular sample differs from the status generally found in average samples (e.g., healthy samples or average diseased samples). Examples include mutated, elevated, decreased, present, absent, etc. Reference to a biomarker with an “elevated status” means that one or more of the above characteristics (e.g., expression or mRNA level) is higher than normal levels. Generally, this means an increase in the characteristic (e.g., expression or mRNA level) as compared to an index value. Conversely reference to a biomarker's “low status” means that one or more of the above characteristics (e.g., gene expression or mRNA level) is lower than normal levels. Generally, this means a decrease in the characteristic (e.g., expression) as compared to an index value. In this context, a “negative status” of a biomarker generally means the characteristic is absent or undetectable.

### Genotyping Assays/Biomarker Analysis

Any suitable genotyping assay and/or method of detecting, analyzing, or otherwise studying the herein biomarkers is contemplated. For example, the genotyping assay can be a restriction fragment length polymorphism identification (RFLPI) of the DNA sample, a random amplified polymorphic detection (RAPD) of the DNA sample, an amplified fragment length polymorphism (AFLPD) of the DNA sample, a polymerase chain reaction (PCR) of the DNA sample, DNA sequencing of the DNA sample, or hybridization of the DNA sample to a nucleic acid microarray.

In some embodiments, the biomarkers related to increased or decreased level of expression of transcripts (i.e., mRNA levels). Methods of measuring or detecting transcript levels are known in the art.

Various technologies are well-known in the art for deducing and/or measuring and/or detecting the levels of one or more transcripts in a cell. Such methods include hybridization-or sequence-based approaches. Hybridization-based approaches typically involve incubating fluorescently labelled cDNA with custom-made microarrays or commercial high-density oligo microarrays. Specialized microarrays have also been designed; for example, arrays with probes spanning exon junctions can be used to detect and quantify distinct spliced isoforms. Genomic tiling microarrays that represent the genome at high density have been constructed and allow the mapping of transcribed regions to a very high resolution, from several base pairs to ˜100 bp. Hybridization-based approaches are high throughput and relatively inexpensive, except for high-resolution tiling arrays that interrogate large genomes. However, these methods have several limitations, which include: reliance upon existing knowledge about genome sequence; high background levels owing to cross-hybridization; and a limited dynamic range of detection owing to both background and saturation of signals. Moreover, comparing expression levels across different experiments is often difficult and can require complicated normalization methods.

In contrast to microarray methods, sequence-based approaches directly determine the cDNA sequence. Initially, Sanger sequencing of cDNA or EST libraries was used, but this approach is relatively low throughput, expensive and generally not quantitative. Tag-based methods were developed to overcome these limitations, including serial analysis of gene expression (SAGE), cap analysis of gene expression (CAGE), and massively parallel signature sequencing (MPSS). These tag-based sequencing approaches are high throughput and can provide precise, digital gene expression levels. However, most are based on Sanger sequencing technology, and a significant portion of the short tags cannot be uniquely mapped to the reference genome. Moreover, only a portion of the transcript is analyzed and isoforms are generally indistinguishable from each other. These disadvantages limit the use of traditional sequencing technology in measuring or detection mRNA levels.

The present methods can also involve a larger-scale analysis of mRNA levels, e.g., the detection of a plurality of biomarkers (e.g., 2-10, or 5-50, or 10-100, or 50-500 or more at one time). In addition, the methods described here can also involve the step of conducting a transcriptomic analysis (i.e., the analysis of the complete set of transcripts in a cell, and their quantity, for a specific developmental stage or physiological condition). Understanding the transcriptome is can be important for interpreting the functional elements of the genome and revealing the molecular constituents of cells and tissues, and also for understanding development and disease and how the biomarkers disclosed herein are indicative or predictive of a particular condition (e.g., LM or LMS). The key aims of transcriptomics are: to catalogue all species of transcript, including mRNAs, non-coding RNAs and small RNAs; to determine the transcriptional structure of genes, in terms of their start sites, 5′ and 3′ ends, splicing patterns and other post-transcriptional modifications; and to quantify the changing expression levels of each transcript during development and under different conditions.

Recently, the development of novel high-throughput DNA sequencing methods has provided a new method for both mapping and quantifying transcriptomes. This method, termed RNA-Seq (RNA sequencing), has advantages over existing approaches for determining transcriptomes.

RNA-Seq uses deep-sequencing technologies. In general, a population of RNA (total or fractionated, such as poly(A)+) is converted to a library of cDNA fragments with adaptors attached to one or both ends. Each molecule, with or without amplification, is then sequenced in a high-throughput manner to obtain short sequences from one end (single-end sequencing) or both ends (pair-end sequencing). The reads are typically 30-400 bp, depending on the DNA-sequencing technology used. In principle, any high-throughput sequencing technology can be used for RNA-Seq, e.g., the Illumina IG18, Applied Biosystems SOLiD22 and Roche 454 Life Science systems have already been applied for this purpose. The Helicos Biosciences tSMS system is also appropriate and has the added advantage of avoiding amplification of target cDNA. Following sequencing, the resulting reads are either aligned to a reference genome or reference transcripts, or assembled de novo without the genomic sequence to produce a genome-scale transcription map that consists of both the transcriptional structure and/or level of expression for each gene.

Further reference can be made regarding transcriptome analysis and RNA-Seq technologies known in the art: (1) Wang et al., Nat Rev Genet. 2009 January; 10(1): 57-63; (2) Lee et al., Circ Res. 2011 Dec. 9; 109(12):1332-41; (3) Nagalakshimi et al., Curr Protoc Mol Biol. 2010 January; Chapter 4:Unit 4.11.1-13; and (4) Mutz et al., Curr Opin Biotechnol. 2013 February; 24(1):22-30, each of which are incorporated herein by reference.

Transcriptome analysis by next-generation sequencing (RNA-seq) allows investigation of a transcriptome at unsurpassed resolution. One major benefit is that RNA-seq is independent of a priori knowledge on the sequence under investigation.

The transcriptome can be profiled by high throughput techniques including SAGE, microarray, and sequencing of clones from cDNA libraries. For more than a decade, oligo-nucleotide microarrays have been the method of choice providing high throughput and affordable costs. However, microarray technology suffers from well-known limitations including insufficient sensitivity for quantifying lower abundant transcripts, narrow dynamic range and biases arising from non-specific hybridizations. Additionally, microarrays are limited to only measuring known/annotated transcripts and often suffer from inaccurate annotations. Sequencing-based methods such as SAGE rely upon cloning and sequencing cDNA fragments. This approach allows quantification of mRNA abundance by counting the number of times cDNA fragments from a corresponding transcript are represented in a given sample, assuming that cDNA fragments sequenced contain sufficient information to identify a transcript. Sequencing-based approaches have a number of significant technical advantages over hybridization-based microarray methods. The output from sequence-based protocols is digital, rather than analog, obviating the need for complex algorithms for data normalization and summarization while allowing for more precise quantification and greater ease of comparison between results obtained from different samples. Consequently the dynamic range is essentially infinite, if one accumulates enough sequence tags. Sequence-based approaches do not require prior knowledge of the transcriptome and are therefore useful for discovery and annotation of novel transcripts as well as for analysis of poorly annotated genomes. However, until recently the application of sequencing technology in transcriptome profiling has been limited by high cost, by the need to amplify DNA through bacterial cloning, and by the traditional Sanger approach of sequencing by chain termination.

The next-generation sequencing (NGS) technology eliminates some of these barriers, enabling massive parallel sequencing at a high but reasonable cost for small studies. The technology essentially reduces the transcriptome to a series of randomly fragmented segments of a few hundred nucleotides in length. These molecules are amplified by a process that retains spatial clustering of the PCR products, and individual clusters are sequenced in parallel by one of several technologies. Current NGS platforms include the Roche 454 Genome Sequencer, Illumina's Genome Analyzer, and Applied Biosystems' SOLiD. These platforms can analyze tens to hundreds of millions of DNA fragments simultaneously, generate giga-bases of sequence information from a single run, and have revolutionized SAGE and cDNA sequencing technology. For example, the 3′ tag Digital Gene Expression (DGE) uses oligo-dT priming for first strand cDNA synthesis, generates libraries that are enriched in the 3′ untranslated regions of polyadenylated mRNAs, and produces base cDNA tags.

In various embodiments the use of such sequencing technologies does not require the preparation of sequencing libraries. However, in certain embodiments the sequencing methods contemplated herein requires the preparation of sequencing libraries.

Any method for making high-throughput sequencing libraries can be used. An example of sequencing library preparation is described in U.S. Patent Application Publication No. 2013/0203606, which is incorporated by reference in its entirety. In some embodiments, this preparation may take the coagulated portion of the sample from the droplet actuator as an assay input. The library preparation process is a ligation-based process, which includes four main operations: (a) blunt-ending, (b) phosphorylating, (c) A-tailing, and (d) ligating adaptors. DNA fragments in a droplet are provided to process the sequencing library. In the blunt-ending operation (a), nucleic acid fragments with 5′- and/or 3′-overhangs are blunt-ended using T4 DNA polymerase that has both a 3′-5′ exonuclease activity and a 5′-3′ polymerase activity, removing overhangs and yielding complementary bases at both ends on DNA fragments. In some embodiments, the T4 DNA polymerase may be provided as a droplet. In the phosphorylation operation (b), T4 polynucleotide kinase may be used to attach a phosphate to the 5′-hydroxyl terminus of the blunt-ended nucleic acid. In some embodiments, the T4 polynucleotide kinase may be provided as a droplet. In the A-tailing operation (c), the 3′ hydroxyl end of a dATP is attached to the phosphate on the 5′-hydroxyl terminus of a blunt-ended fragment catalyzed by exo-Klenow polymerase. In the ligating operation (d), sequencing adaptors are ligated to the A-tail. T4 DNA ligase is used to catalyze the formation of a phosphate bond between the A-tail and the adaptor sequence. In some embodiments involving cfDNA, end-repairing (including blunt-ending and phosphorylation) may be skipped because the cfDNA are naturally fragmented, but the overall process upstream and downstream of end repair is otherwise comparable to processes involving longer strands of DNA.

In another example, sequencing library preparation can involve the production of a random collection of adapter-modified DNA fragments (e.g., polynucleotides) that are ready to be sequenced. Sequencing libraries of polynucleotides can be prepared from DNA or RNA, including equivalents, analogs of either DNA or cDNA, for example, DNA or cDNA that is complementary or copy DNA produced from an RNA template, by the action of reverse transcriptase. The polynucleotides may originate in double-stranded form (e.g., dsDNA such as genomic DNA fragments, cDNA, PCR amplification products, and the like) or, in certain embodiments, the polynucleotides may originated in single-stranded form (e.g., ssDNA, RNA, etc.) and have been converted to dsDNA form.

By way of illustration, in certain embodiments, single stranded mRNA molecules may be copied into double-stranded cDNAs suitable for use in preparing a sequencing library. The precise sequence of the primary polynucleotide molecules is generally not material to the method of library preparation, and may be known or unknown. In one embodiment, the polynucleotide molecules are DNA molecules. More particularly, in certain embodiments, the polynucleotide molecules represent the entire genetic complement of an organism or substantially the entire genetic complement of an organism, and are genomic DNA molecules (e.g., cellular DNA, cell free DNA (cfDNA), etc.), that typically include both intron sequence and exon sequence (coding sequence), as well as non-coding regulatory sequences such as promoter and enhancer sequences. In certain embodiments, the primary polynucleotide molecules comprise human genomic DNA molecules, e.g., cfDNA molecules present in peripheral blood of a subject.

Preparation of sequencing libraries for some NGS sequencing platforms is facilitated by the use of polynucleotides comprising a specific range of fragment sizes. Preparation of such libraries typically involves the fragmentation of large polynucleotides (e.g. cellular genomic DNA) to obtain polynucleotides in the desired size range.

Methods and further information regarding purification, processing, sequence, and analyzing cfDNA can be found in the following references, each of which are incorporated herein by reference:

Yiğit B, Boyle M, Özler O, et al. “Plasma cell-free DNA methylation: a liquid biomarker of hepatic fibrosis,” Gut, Published Online 20 Jan. 2018;

Lehmann-Werman R et al., “Identification of tissue-specific cell death using methylation patterns of circulating DNA,” Proc Natl Acad Sci USA 2016; 113:E1826-34;

Hardy T et al., “Plasma DNA methylation: a potential biomarker for stratification of liver fibrosis in non-alcoholic fatty liver disease,” Gut ,2016, Epub ahead of print 21 Mar. 2016;

Cui M et al., “Cell-Free circulating DNA: a new biomarker for the acute coronary syndrome,” Cardiology 2013; 124:76-84;

Zhong X Y et al., “Increased concentrations of antibody-bound circulatory cell-free DNA in rheumatoid arthritis,” Clin Chem 2007; 53: 1609-14;

Bartoloni E et al., “Increased levels of circulating DNA in patients with systemic autoimmune diseases: a possible marker of disease activity in Sjögren's syndrome,” Lupus 2011; 20:928-35;

Rainer T H, Wong L K, Lam W, Yuen E, Lam N Y, Metreweli C, et al. Prognostic use of circulating plasma nucleic acid concentrations in patients with acute stroke. Clin Chem 2003; 49:562-9. Google Scholar;

Antonatos D, Patsilinakos S, Spanodimos S, Korkonikitas P, Tsigas D. Cell-free DNA levels as a prognostic marker in acute myocardial infarction. Ann N Y Acad Sci 2006; 1075:278-81. Google Scholar;

Jiang P, Lo Y M. The long and short of circulating cell-free DNA and the Ins and outs of molecular diagnostics. Trends Genet 2016; 32:360-71. Google Scholar;

Wou K, Feinberg J L, Wapner R J, Simpson J L. Cell-free DNA versus intact fetal cells for prenatal genetic diagnostics: what does the future hold? Expert Rev Mol Diagn 2015; 15:989-98. Google Scholar;

Benn P. Non-invasive prenatal testing using cell free DNA in maternal plasma: recent developments and future prospects. J Clin Med 2014; 3:537-65. Google Scholar;

Bianchi D W, Chudova D, Sehnert A J, Bhatt S, Murray K, Prosen T L, et al. Noninvasive prenatal testing and incidental detection of occult maternal malignancies. J Am Med Assoc 2015; 314: 162-9. Google Scholar;

Hahn S, Rusterholz C, Hösli I, Lapaire O. Cell-free nucleic acids as potential markers for preeclampsia. Placenta 2011; 32(Suppl): S17-20. Google Scholar;

Ryan W L. Method and device for collecting and preserving cells for analysis. U.S. Patent Application 2010; 0317107:A1. Google Scholar;

Holmes E E, Jung M, Meller S, Leisse A, Sailer V, Zech J, et al. Performance evaluation of kits for bisulfite-conversion of DNA from tissues, cell lines, FFPE tissues, aspirates, lavages, effusions, plasma, serum, and urine. PLoS One 2014; 9:e93933. Google Scholar;

Frommer M, McDonald L E, Millar D S, Collis C M, Watt F, Grigg G W, et al. A genomic sequencing protocol that yields a positive display of 5-methylcytosine residues in individual DNA strands. Proc Natl Acad Sci USA 1992; 89:1827-31. Google Scholar;

Holmes E E, Jung M, Meller S, Leisse A, Sailer V, Zech J, et al. Performance evaluation of kits for bisulfite-conversion of DNA from tissues, cell lines, FFPE tissues, aspirates, lavages, effusions, plasma, serum, and urine. PLoS One 2014; 9:e93933. Google Scholar;

Nucleic acids may also be characterized by amplification (for example by conventional polymerase chain reaction). Other methods of determining the sequence of DNA and/or RNA known in the art such as nanopore sequencing, sequencing by ligation (sometimes known as SOLid), combinatorial probe anchor synthesis, pyrosequencing, ion torrent sequencing, or sequencing by synthesis (for example Illumina's Next Generation Sequencing technologies). Such sequencing methods may be usefully directed at known oncogenes (genes where upregulation or dysregulation are known to be associated with malignancy or with diagnostic, prognostic or predictive value in malignant tissues) in order to enrich for data likely to be useful for discriminating between benign and malignant uterine neoplasms such as LM and LMS.

In some embodiment, the analytical methods for detecting genotypes can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication No. WO 99/36760) and PCT/US01/04285 (International Publication No. WO 01/58593), which are all incorporated herein by reference in their entirety for all purposes.

The present invention also contemplates sample preparation methods in certain preferred embodiments. Prior to or concurrent with genotyping, the genomic sample may be amplified by a variety of mechanisms, some of which may employ PCR. See, for example, PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. Ser. No. 09/513,300, which are incorporated herein by reference.

Other suitable amplification methods include the ligase chain reaction (LCR) (for example, Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. Ser. Nos. 09/916,135, 09/920,491 (U.S. Patent Application Publication 20030096235), Ser. No. 09/910,292 (U.S. Patent Application Publication 20030082543), and Ser. No. 10/013,598.

The sequence data generated as described herein can be analyzed by using software to detect mutation characteristics of the neoplasms genotypic signature and distinguish these from germline mutations such as the Illumina Somatic Variant Caller, Pisces or similar algorithms suitable for the detection of point mutations, including single nucleotide polymorphisms and single base insertion and deletions. Short multinucleotide variants (MNVs) can also be detected by algorithms known in the art such as Illumina's Scylla and the Broad Institutes GATK. Larger genetic changes such as copy number variants (CNVs) or structural variants can be detected using algorithmic implementations such as GATK, MANTA, GenomeS TRIP or Illumina's “CNV Robust Analysis For Tumors” known as CRAFT, or other computational tools for copy number variation detection such as are known in the art.

With respect to the qualitative analysis of the transcriptomic data, splice variants of RNA can be detected in sequence data using software such as the CLASS2 algorithm, Illumina's RNA Splice Variant Caller, GATK or other methods as described in Hooper (2014). Quantitative transcriptomic data may also be addressed using software such as Empirical Analysis of Digital Gene Expression Data in R (edgeR), DESeq2 or Limma Structural variants in RNA, including RNA fusions, can also be detected using software such as MANTA while the empiric expression of the resulting “chimeric” proteins can be confirmed by direct detection of the proteins in situ by methods known in the art such as immunohistochemistry to localize specific protein epitopes and chromogenic in situ hybridization or fluorescent in situ hybridization to localize specific nucleic acid signatures.

Once the genotypic and/or transcriptomic profile of the biological samples in question has been obtained by the methods described above, extracting nucleic acids, sequencing nucleic acids and subjecting sequence data to analyses to detect various differential genetic signatures, e.g., single nucleotide variants, multinucleotide variants (CNVs), copy number variants (CNVs) and in the case of RNA splice variants, RNA fusions, differential expression levels, differential epigenetic characteristic (e.g., differential methylation patterns), each of which is a potential biomarker. The data generated may then be compared to reference data sets representing the genotypic and transcriptomic profiles of confirmed healthy tissues, or confirmed LMS tissues, or confirmed LM-only tissues (i.e., no LMS cells lurking therein). In some embodiments the data sets can be augmented to include extrinsic data such as patient demographics, ancestry, medical history and risk factors that those skilled in the art will appreciate might contribute independent inferential value to the multivariate data set comprising genotypic and transcriptomic data. In some embodiments this comparison may be effected by implementing the reference data sets in a tool, device or piece of software that provides a means of partitioning the variance in the data matrix defined by the status of each biomarker for each reference datum in a manner that most efficiently partitions the data into a number of orthogonal eigenvectors that is significantly fewer than the number of biomarkers, such as factor analysis or principal components analysis. The known disease status of the reference data can then be projected into the space defined by the principle eigenvectors or principal components and where different disease states (for example leiomyoma and leiomyosarcoma) occupy discrete volumes of that space the subject's data profile can be projected into the same space and an inference made as to whether the subject shares a profile with one or other disease state or with neither. Alternately, unsupervised hierarchical cluster analysis can be used to determine clusters of similar data, the data clusters can be evaluated post-hoc for correspondence with a particular disease state and the tendency of a subject's profile to fall within a cluster associated with a disease state can be used to evaluate the likelihood or probability that the subject's biological sample is one disease state (e.g., LM) or the other (e.g., LMS). In both principal components or cluster analysis methods can be used to evaluate the robustness of the structural features of the reference data sets as well as the confidence with which the subject's data can be assigned to one or the disease state.

Another family of approaches to the comparison of the reference data to the subject data is to allow supervised multivariate reduction of the reference data set such as canonical variates analysis or discriminant function analysis where the known disease status of each reference datum is first used to derive the multivariate descriptor that best discriminates between the disease states of interest and then the subject data is projected into the discriminant space in order to generate a probability of classification into one or other disease state. Methods such as bootstrap analyses and cross validation may be used to evaluate the robustness of the multivariate solution and the specific classification of the subject with respect to the disease state. In some embodiments the disease states so referenced are benign and malignant uterine neoplasms. In some embodiments the disease states so referenced are leiomyoma and uterine sarcoma. In some embodiments the disease states so referenced are leiomyoma and leiomyosarcoma. In some embodiments the tool or device comprises templates for data entry, implementations of data quality control methods, reference data sets, recommended analytical procedures, clinician elected analytical options, standardized data output templates and automatically generated recommended inferential prose statements to assist the analyst in understanding and communicating the resulting risk evaluation to other members of the clinical team and to the subject.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, for example Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Ser. Nos. 10/197,621, 10/063,559 (U.S. Publication No. 20020183936), Ser. Nos. 10/065,856, 10/065,868, 10/328,818, 10/328,872, 10/423,403, and 60/482,389.

### Methods of Use

Disclosed herein are improved methods for the early preoperative diagnosis of myometrial tumors. In one aspect, the methods described herein provide a means to detect whether a myometrial tumor comprises a leiomyosarcoma to help prevent accidental malignant dissemination derived from surgical methods like morcellation. In another aspect, the methods described herein provide a means to diagnosis a myometrial tumor for the presence of leiomyomas, or leiomyosarcomas, or both leiomyomas and leiomyosarcomas.

It will be appreciated that surgery remains the long-term therapeutic option for uterine leiomyoma. Specifically, laparoscopic myomectomy with morcellation is the gold standard intervention for women who wish to preserve their fertility. However, this surgery carries potential detrimental effects for patients with undiagnosed occult leiomyosarcoma (LMS). Once the genotype of uterine leiomyoma or leiomyosarcoma through tissue and/or liquid biopsy is confirmed, morcellation may be avoided since it has the chance to spread an otherwise hidden malignant tumor.

Thus, in one embodiment, the present disclosure provides a method for diagnosing a myometrial tumor in a subject, comprising a unique integrative molecular analysis of transcriptomic and genomic data on a biological sample (tumoral tissue) from the subject, to determine whether the subject has a LM, LMS and/or IMT profile. Thus, various aspects of the disclosure relate to an initial diagnostic screen of the myometrial tumors (from tumoral tissue or another biological sample, such as a blood- or plasma-based based test) to ensure that there is no detection of leiomyosarcoma tissue.

Thus, in one aspect, the disclosure provides a method for diagnosing a myometrial tumor in a subject, comprising a unique integrative molecular analysis of transcriptomic and genomic data on a biological sample (e.g., tumoral tissue) from the subject, to determine whether the subject has a LM, LMS and/or IMT profile and/or genotype.

In another aspect, the disclosure provides a method for diagnosing a myometrial tumor in a subject, comprising performing a genotyping assay on a biological sample from the subject to determine whether the subject has a LM genotype. In various embodiments, the genotyping assay involved the detection of one or more biomarkers that are indicative of LM.

In still another aspect, the disclosure provides a method for diagnosing a myometrial tumor in a subject, comprising performing a genotyping assay on a biological sample from the subject to determine whether the subject has an LMS genotype. In various embodiments, the genotyping assay involved the detection of one or more biomarkers that are indicative of LMS.

In yet another aspect, the disclosure provides a method for diagnosing a myometrial tumor in a subject, comprising performing a genotyping assay on a biological sample from the subject to determine whether the subject has an IMT genotype. In various embodiments, the genotyping assay involved the detection of one or more biomarkers that are indicative of IMT. In other aspect, the methods for diagnosing LM, LMT, or IMT can be combined with a therapeutic method or treatment step for treating a myometrial tumors/uterine neoplasm (e.g., a leiomyoma or leiomyosarcoma).

In another embodiment, the present disclosure provides a method for treating a myometrial tumor comprising first confirming with a genotyping assay that the tumor does not contain a leiomyosarcoma and then surgically remove the myometrial tumor.

In still other embodiments, the disclosure provides a method for treating a uterine leiomyoma in a subject, comprising: (a) performing a genotyping assay on a biological sample from the subject to determine whether the subject has a uterine leiomyosarcoma genotype, and (b) surgically removing the uterine leiomyoma if the subject does not have a uterine leiomyosarcoma genotype.

Thus, various aspects of the disclosure relate to a new and improved method of morcellation-based treatment of non-cancerous leiomyomas (i.e., those that are determined to be free of leiomyosarcomas) involving an initial diagnostic screen of the leiomyomas tissue (or another biological sample, such as a blood- or plasma-based based test) to ensure that there is no detection of leiomyosarcoma tissue.

Thus, in another embodiment, the present disclosure provides a method for detecting the presence of uterine leiomyosarcoma in a uterine leiomyoma in a subject, comprising performing a genotyping assay on a biological sample from the subject to determine whether the subject has a uterine leiomyosarcoma genotype.

In various embodiments, the methods may also include detecting or confirming the presence a uterine leiomyoma in a sample.

In various preferred embodiments, the biological sample which is analyzed is a blood sample. In other embodiments, the biological sample which is analyzed is a plasma sample.

In various embodiments, the detection of a leiomyosarcoma genotype in a sample involves the detection of one or more biomarkers from Tables 3, 6, 7, and 8. The genotyping assay may involve biomarkers from only one table, or from a combination of tables. For example, one or more biomarkers from Table 3 may be combined with one or more biomarkers from Tables 6, 7, or 8. In another embodiments, one or more biomarkers from Table 6 may be combined with one or more biomarkers from Tables 3, 7, or 8. In still other embodiments, one or more biomarkers from Table 7 may be combined with one or more biomarkers from Tables 3, 6, or 8. In other embodiments, one or more biomarkers from Table 8 may be combined with one or more biomarkers from Tables 3, 6, or 7. In still other embodiments, any first biomarker disclosed herein in any table or listing or set may be combined with any second biomarker disclosed. Further, this combination may be combined with any third, or fourth, or fifth, or sixth, or seventh, or eighth, or ninth, or tenth or more biomarkers disclosed anywhere herein. Such combinations of biomarkers for the detection of LM and/or LMS may be referred to biomarker signatures.

In some embodiments, a biomarker is differentially expressed in a sample from a subject that has a malignant uterine tumor compared to a sample from a subject that does not have a malignant uterine tumor, or a subject that has benign uterine neoplasms. In some embodiments, a biomarker is differentially expressed in a sample from a subject that has leiomyosarcoma compared to a sample from a subject that does not have leiomyosarcoma, or a subject that has leiomyoma. In some embodiments, a biomarker is differentially expressed in a sample from a subject that has leiomyoma compared to a sample from a subject that does not have leiomyoma, or a subject that has leiomyosarcoma.

In some embodiments, the biomarker signature indicative of leiomyosarcoma (LMS) comprises one or more biomarkers (e.g., or 2, 3, 4, 5, 6, 7, 8, 9, or 10 or more biomarkers) selected from the group consisting of FGF8, RET, PTEN, ATM, CADM1, KMT2A, NOTC H2, MCL1, DDR2, CCND1, FGF19, FGF3, MDM4, KRAS, SDCCAG8, CCND2, RP11-61102.2, MDM2, ARID1A, FGF14, LAMP1, NA, FGF9, FLT1, ALOX5AP, BRCA2, RB1, MYCL, MPL, HPDL, MUTYH, RAD54L, RAD51B, FANCI, TSC2, PALB2, NLRC3, SLX4, CREBBP, CDH1, RP11-525K10.1, RAP1GAP2, RAD51L3- RFFL, ERBB2, BRCA1, TEX14, RPS6KB1, TP53, RBFOX3, BCL2, STK11, NOTCH3, JAK3, TGFBR3, CCNE1, AKT2, ERCC2, PPP1R13L, PIK3CD, GNAS, ERG, ERBB4, BARD1, RNA5SP495, CHEK2, RP1-302D9.3, EP300, RNU6-688P, MSH6, VHL, MKRN2, ATR, MLH1, TET2, FGF2, FGFR3, PDGFRA, KDR, FGF5, APC, HMGX B3, CSF1R, PDGFRB, FGF10, PIK3R1, DHFR, RO S1, HIVEP1, ESR1, BYSL, MET, SMO, BRAF, DPP 6, CARD11, EGFR, CASC11, NRG1, FGFR1, NOTCH1, MLLT3, LINGO2, PTCH1, and AR.

In some embodiments, the biomarker signature indicative of leiomyosarcoma (LMS) comprises one or more biomarkers (e.g., or 2, 3, 4, 5, 6, 7, 8, 9, or 10 or more biomarkers) selected from the group consisting of CDK4, FGF10, FGF5, MYC, MYCL1, NRG1, FGF1, FGF14, JAK2, KRAS, FGF7, MDM4, FGF5, RET, ALK, BRCA2, FGFR3, FGFR4, FLT3, NTRK1, PAX3, PAX7, RET, ROS1, and TMPRSS2. In some embodiments, the biomarker signature indicative of leiomyosarcoma comprises one or more biomarkers (e.g., or 2, 3, 4, 5, 6, 7, 8, 9, or 10 or more biomarkers) selected from the group consisting of ALK, BRCA2, FGFR3, FGFR4, FLT3, NTRK1, PAX3, PAX7, RET, ROS1, and TMPRSS2. In some embodiments, the biomarker signature indicative of leiomyosarcoma comprises upregulation of one or more biomarkers (e.g., or 2, 3, 4, 5, 6, 7, 8, 9, or 10 or more biomarkers) selected from the group consisting of ALK, BRCA2, FGFR3, FGFR4, FLT3, NTRK1, PAX3, PAX7, RET, ROS1, and TMPRSS2.

In some embodiments, the biomarker signature indicative of leiomyosarcoma (LMS) comprises one or more biomarkers (e.g., or 2, 3, 4, 5, 6, 7, 8, 9, or 10 or more biomarkers) selected from the group consisting of ALK, BARD1, BRCA2, CCNE1, CDK4, FGF1, FGF10, FGF5, FGFR3, FLT3, JAK2, KRAS, NTRK1, PAX3, PAX7, PTEN, RET, ROS1, and MPRSS2. In some embodiments, the biomarker signature indicative of leiomyosarcoma comprises mRNA upregulation in one or more biomarkers (e.g., or 2, 3, 4, 5, 6, 7, 8, 9, or 10 or more biomarkers) selected from the group consisting of ALK, BRCA2, FGFR3, FGFR4, FLT3, NTRK1, PAX3, PAX7, RET, ROS1, and MPRSS2. In some embodiments, the biomarker signature indicative of leiomyosarcoma comprises deletion (partial or complete) of one or more biomarkers selected from the group consisting of FGF1, JAK2, KRAS, and PTEN. In some embodiments, the biomarker signature indicative of leiomyosarcoma comprises duplication of one or more biomarkers selected from the group consisting of CDK4, FGF10, and FGF5. In some embodiments, the biomarker signature indicative of leiomyosarcoma comprises a mutation in one or more biomarkers selected from the group consisting of BARD1, CCNE1, FGF5, and RET. In some embodiments, the mutation is a single nucleotide polymorphism (SNP).

In some embodiments, the biomarker signature indicative of leiomyosarcoma (LMS) comprises a copy number variant (CNV) duplication in one or more biomarkers selected from the group consisting of CDK4, FGF10, FGF5, MYC, MYCL1, and NRG1. In some embodiments, the gene signature indicative of leiomyosarcoma comprises a CNV deletion in one or more biomarkers selected from the group consisting of FGF1, FGF14, JAK2, and KRAS. In some embodiments, the biomarker signature indicative of leiomyosarcoma comprises a CNV duplication and deletion in one or more biomarkers selected from the group consisting of FGF14, FGF7, MDM4, MYCL1, and NRG1.

In some embodiments, the biomarker signature indicative of leiomyosarcoma (LMS) comprises a single nucleotide variant (SNV) in one or more biomarkers selected from the group consisting of FGFS and RET. In some embodiments, the gene signature indicative of leiomyosarcoma comprises mRNA upregulation in one or more biomarkers (e.g., or 2, 3, 4, 5, 6, 7, 8, 9, or 10 or more biomarkers) selected from the group consisting of ALK, BRCA2, FGFR3, FGFR4, FLT3, NTRK1, PAX3, PAX7, RET, ROS1, and TMPRSS2.

In some embodiments, the biomarker signature indicative of leiomyoma (LM) comprises one or more biomarkers (e.g., or 2, 3, 4, 5, 6, 7, 8, 9, or 10 or more biomarkers) selected from the group consisting of FGFR2, KLLN, PTEN, ATM, KMT2A, MTOR, NRAS, NOTCH2, FGF19, AP001888.1, FGF3, MRE11A, MDM4, PTPN11, SDCCAG8, FGF6, ERBB3, MDM2, NA, LAMP1, FGF9, FLT1, BRCA2, MYCL, RP11-982M15.2, MPL, HPDL, SLC35F4, RAD51B, RAD51, IDH2, TSC2, SLX4, CREBBP, RAD51L3-RFFL, TP53, RBFOX3, STK11, NOTCH3, TGFBR 3, AKT2, GNAS-AS1, ERG, MYCNOS, BARD1, EP300, DNMT3A, MSH2, MSH6, VHL, RAF1, PIK3CB, PIK3CA, TFRC, MLH1, BAP1, TET2, FGFR3, PDGFRA, MRPS18C, APC, HMGXB3, CSF1R, PDGFRB, FGFR4, FGF10, ESR1, BYSL, CCND3, SMO, DPP6, EGFR, CDK6, MYC, NRG1, NOTCH1, MLLT3, RP11-145E5.5, JAK2, GNAQ, PTCH1, and AR.

In some embodiments, the biomarker signature indicative of leiomyoma (LM) comprises one or more biomarkers selected from the group consisting of CCND1, FGFR3, and MET. In some embodiments, the biomarker signature indicative of leiomyoma comprises a CNV duplication in FGFR3. In some embodiments, the biomarker signature indicative of leiomyoma comprises a CNV deletion in MET. In some embodiments, the biomarker signature indicative of leiomyoma comprises a CNV duplication in CCND1 and FGFR3, and a CNV deletion in MET. In some embodiments, the biomarker signature indicative of leiomyoma comprises a CNV duplication in CCND1.

In some embodiments, the biomarker signature indicative of leiomyoma (LM) comprises a T-G mutation in chr12-4551244. In some embodiments, the gene signature indicative of leiomyoma comprises a G-T mutation in chr11-94192599.

In some embodiments, the biomarker signature indicative of leiomyosarcoma (LMS) comprises a C-A mutation in chr10-43597827. In some embodiments, the gene signature indicative of leiomyosarcoma comprises a TAA-T mutation in chr4-81206898.

The methods of the present disclosure may involve morcellation or other surgical methods for removing leiomyomas after they have been determined not to comprise any leiomyosarcomas. It will be well known that large tissue masses, such as fibroid tissue masses (leiomyomas), are traditionally excised during a surgical procedure and removed intact from the patient through the surgical incision. These tissue masses can easily be several centimeters in diameter or larger. In minimally invasive surgery, the surgery is typically conducted using incisions of less than 1 centimeter, and often 5 millimeters or less. Thus, the trend toward the use of minimally invasive surgery has created a need to reduce large tissue masses to a size small enough to fit through an opening which may be 1 centimeter or smaller in size. It will be appreciated that one common procedure for reducing the size of large tissue masses is morcellation.

Morcellation medical devices are well-known in the art. For example, the instruments described in U.S. Pat. Nos. 5,037,379; 5,403,276; 5,520,634; 5,327,896 and 5,443,472 can be used herein (each patent is incorporated herein by reference). As those references illustrate, excised tissue is morcellated (i.e. debulked), collected and removed from the patient's body through, for example, a surgical trocar or directly through one of the surgical incisions.

Mechanical morcellators cut tissue using, for example, sharp end-effectors such as rotating blades. Electrosurgical and ultrasonic morcellators use energy to morcellate tissue. For example, a system for fragmenting tissue utilizing an ultrasonic surgical instrument is described in “Physics of Ultrasonic Surgery Using Tissue Fragmentation”, 1995 IEEE Ultrasonics Symposium Proceedings, pages 1597-1600.

In some embodiments, it may be desirable to conduct the morcellation in conjunction with a tissue specimen bag in order to prevent morcellated tissue from spreading to other parts of the body during and after the morcellation procedure. For example, the excised tissue is can be transferred to a specimen bag prior to being morcellated. However some morcellators are used without specimen bags. Specimen bags are, therefore, designed to hold excised tissue without spilling tissue, or tissue components, into the abdominal cavity during morcellation. It will be apparent that specimen bags used with morcellators must be strong enough to prevent tears or cuts which might spill the contents of the specimen bag.

Ultrasonic morcellation instruments may be particularly advantageous for use in certain surgical procedures and for debulking certain types of tissue. A blunt or rounded ultrasonic morcellator tip may reduce the possibility of unintended cutting or tearing of a specimen bag while the ultrasonic energy morcellates the tissue. U.S. Pat. No. 5,449,370, hereby incorporated herein by reference, describes a blunt tipped ultrasonic surgical instrument capable of morcellating tissue contained within a specimen bag.

In some embodiments, a biological sample can be used to define whether it has a leiomyoma or leiomyosarcoma genotype. This preoperative screen can be a tissue and/or liquid biopsy, which in various aspects involves conducting a genotype assay to screen for LMS and/or LM in a liquid biological sample, such as blood or plasma. If the tissue and/or liquid biopsy at least detects a LMS genotype, morcellation could be advised against for treating a leiomyoma.

Any morcellation tools known or described in the art are contemplated here, For example, morcellation tools are described, for example in U.S. Pat. Nos. 9,955,922, 9,877,739, 9,539,018, 9,044,210, 8,308,746, and 6,162,235, each of which is incorporated by reference.

### Kits

The present disclosure also relates to kits and/or packages comprising compositions and/or instructions involving the diagnostic and/or clinical methods described herein.

A “kit” refers to any system for carrying out a method of the invention.

The present disclosure also provides kits and devices for use in measuring the level of a biomarker set as described herein. Such a kit or device can comprise one or more binding agents that specifically bind to a gene product of target biomarkers, such as the biomarkers listed in any of Tables 1-10. For example, such a kit or detecting device may comprise at least one binding agent that is specific to one or more protein biomarkers selected from Tables 1-10. In some instances, the kit or detecting device comprises binding agents specific to two or more members of the protein biomarker set described herein.

Levels of specific expression products of genes (e.g., NUPR1, CADM1, NPAS3, ATP1A1, and/or TRAK1; CRYAB, NFATC2, BMP2, PMAIP1, ZFYVE21, CILP, SLF2, MATN2, and/or FGF7) can be assessed by any appropriate method. In some embodiments, the levels of specific expression products are analyzed using one or more assays comprising any solid support (e.g., one or more chips). For example, a solid support (e.g., a chip) may be used to analyze at least one (e.g., 1, 2, 3, 4, 5, 6, 7, 8, 9, 10, or more) biological sample(s) of or from a subject.

Sections of the solid support (e.g., the chip) may be modified with one binding partner or more than one binding partner. The solid support may be linked in any manner to the binding partner(s). As a non-limiting example, the binding partner(s) may be bound (e.g., bound directly) onto the surface of the solid support or covalently linked through appropriate coupling chemistry in any manner including, but not limited to: linkage through a epoxide on the surface, creation of an amido link (i.e., through NHS EDC chemistry) using a amine or carboxylic acid group present on the surface, linkage between a thiol and a thiol reactive group (i.e., a maleimide group), formation of a Schiff base between aldehyde and amines, reaction to an anhydride present on the surface, and/or through a photo-activatable linker.

The binding partner may be any binding partner useful for the instant compositions or methods. For example, the binding partner may be a protein (with naturally occurring amino acids or artificial amino acids), one or more nucleic acids made of naturally occurring bases or artificial bases (including, for example, DNA or RNA), sugars, carbohydrates, one or more small molecules (including, but not limited to one or more of: a vitamin, hormone, cofactor, heme group, chelate, fatty acid, or other known small molecule, and/or a phage).

The binding partners may be applied to the surface of the substrate by deposition of a droplet at a pre-defined location in any manner and using any device including, but not limiting to: the use of a pipette, a liquid dispenser, plotter, nano-spotter, nano-plotter, arrayer, spraying mechanism or other suitable fluid handling device.

In some embodiments, antibodies or antigen-binding fragments are provided that are suited for use in the instant methods and compositions. Immunoassays utilizing such antibody or antigen-binding fragments useful for the instant compositions and methods may be competitive or non-competitive immunoassays in either a direct or an indirect format. Non-limiting examples of such immunoassays are Enzyme Linked Immunoassays (ELISA), radioimmunoassays (RIA), sandwich assays (immunometric assays), flow cytometry-based assays, western blot assays, immunoprecipitation assays, immunohistochemistry assays, immuno-microscopy assays, lateral flow immuno-chromatographic assays, and proteomics arrays. For example, the binding partners may be antibodies (or antibody-binding fragments thereof) with specificity towards a protein of interest including one or more of unciliated epithelial biomarkers NUPR1, CADM1, NPAS3, ATP1A1, and/or TRAK1; or one or more of stromal biomarkers CRYAB, NFATC2, BMP2, PMAIP1, ZFYVE21, CILP, SLF2, MATN2, and/or FGF7.

In some embodiments, oligonucleotide binding partners are used to assess the levels of specific expression products of genes. The oligonucleotide binding partners may be of any type known or used. As a set of non-limiting examples, in certain embodiments the oligonucleotide probes may be RNA oligonucleotides, DNA oligonucleotides, a mixture of RNA oligonucleotides and DNA nucleotides, and/or oligonucleotides that may be mixtures of RNA and DNA. The oligonucleotide binding partners may be naturally occurring or synthetic. The oligonucleotide binding partners may be of any length. As a set of non-limiting examples, the length of the oligonucleotide binding partners may range from about 5 to about 50 nucleotides, from about 10 to about 40 nucleotides, or from about 15 to about 40 nucleotides. The array may comprise any number of oligonucleotide binding partners specific for each target gene. For example, the array may comprise less than 10 (e.g., 9, 8, 7, 6, 5, 4, 3, 2, or 1) oligonucleotide probes specific for each target gene. As another example, the array may comprise more than 10, more than 50, more than 100, or more than 1000 oligonucleotide binding partners specific for each target gene.

The array may further comprise control binding partners such as, for example mismatch control oligonucleotide binding partners or control antibodies or antigen binding fragments thereof. Where mismatch control oligonucleotide binding partners are present, the quantifying step may comprise calculating the difference in hybridization signal intensity between each of the oligonucleotide binding partners and its corresponding mismatch control binding partner. Where control antibodies or antigen binding fragments thereof are present, the quantifying step may comprise calculating the difference in hybridization signal intensity between antibodies or antigen binding fragments for the genes under examination (e.g., NUPR1, CADM1, NPAS3, ATP1A1, and/or TRAK1; CRYAB, NFATC2, BMP2, PMAIP1, ZFYVE21, CILP, SLF2, MATN2, and/or FGF7) and a control or “housekeeping” antibody or antigen binding fragment thereof. The quantifying may further comprise calculating the average difference in hybridization signal intensity between each of the oligonucleotide probes and its corresponding mismatch control probe for each gene.

The array (e.g., chip) may contain any number of analysis regions. As a set of non-limiting examples, the array may contain one or more than one (e.g., 2, 3, 4, 5, 6, 7, 8, 9, 10, 20, 25, 30, 35, 40, or more) analysis regions. Each analysis region may comprise any number of binding partners immobilized to a substrate portion therein. As a non-limiting set of examples, each analysis region may comprise between one and 1,000 binding partners, one and 500 binding partners, one and 250 binding partners, one and 100 binding partners, two and 1,000 binding partners, two and 500 binding partners, two and 250 binding partners, two and 100 binding partners, three and 1,000 binding partners, three and 500 binding partners, three and 250 binding partners, or three and 100 binding partners immobilized to a substrate portion therein.

Binding partners including, but not limited to, antibodies or antigen-binding fragments that bind to the specific antigens of interest can be immobilized, e.g., by binding to a solid support (e.g., a chip, carrier, membrane, columns, proteomics array, etc.). In one set of embodiments, a material used to form the solid support has an optical transmission of greater than 90% between 400 and 800 nm wavelengths of light (e.g., light in the visible range). Optical transmission may be measured through a material having a thickness of, for example, about 2 mm (or in other embodiments, about 1 mm or about 0.1 mm). In some instances, the optical transmission is greater than or equal to 80%, greater than or equal to 85%, greater than or equal to 88%, greater than or equal to 92%, greater than or equal to 94%, or greater than or equal to 96% between 400 and 800 nm wavelengths of light. In some embodiments, the material used to form the solid support has an optical transmission of less than or equal to 99.9%, less than or equal to 96%, less than or equal to 94%, less than or equal to 92%, less than or equal to 90%, less than or equal to 85%, less than or equal to 80%, less than or equal to 50%, less than or equal to 30%, or less than or equal to 10% between 400 and 800 nm wavelengths of light. Combinations of the above-referenced ranges are also possible.

The array may be fabricated on a surface of virtually any shape (e.g., the array may be planar) or even a multiplicity of surfaces. Non-limiting examples of solid support materials useful for the compositions and methods described herein may include glass, plastics, elastomeric materials, membranes, or other suitable materials for performing immunoassays. The solid support may be formed from one material, or it may be formed from two or more materials.

Specific solid support materials may include, but are not limited to: any type of glass (e.g., fused silica, borosilicate glass, Pyrex®, or Duran®). In one embodiment, the solid support is a glass chip. The solid support may also comprise a non-glass substrate (e.g., a plastic substrate) coated with a glass film dioxide produced by a process such as sputtering, oxidation of silicon, or through reaction of silane reagents. The glass surface may be further modified with functionalized silane reagents including, for example: amine-terminated silanes (aminopropyltriethoxy silane) and epoxide-terminated silanes (glycidoxypropyltrimethoxysilane).

Additional specific solid support materials may include, but are not limited to: thermoplastic polymers and may comprise one or more of: polystyrene, polycarbonate, polymethylmetacrylate, cyclic olefin copolymers, polyethylene, polypropylene, polyvinyl chloride, polyvinylidene difluoride, any fluoropolymers (e.g., polytetrafluoroethylene, also known as Teflon®), polylactic acid, poly(methyl methacrylate) (also known as PMMA or acrylic; e.g., Lucite®, Perspex®, and Plexiglas®), and acrylonitrile butadiene styrene.

Additional specific solid support materials may include, but are not limited to: one or more elastomeric materials including polysiloxanes (silicones such as polydimethylsiloxane) and rubbers (polyisoprene, polybutadiene, chloroprene, styrene-butadiene, nitrile rubber, polyether block amides, ethylene-vinyl acetate, epichlorohydrin rubber, isobutene-isoprene, nitrile, neoprene, ethylene-propylene, and hypalon).

Additional specific solid support materials may include, but are not limited to: one or more membrane substrates such as dextran, amyloses, nylon, Polyvinylidene fluoride (PVDF), fiberglass, and natural or modified celluloses (e.g., cellulose, nitrocellulose, CNBr-activated cellulose, and cellulose modified with polyacrylamides, agaroses, and/or magnetite). The nature of the support can be either fixed or suspended in a solution (e.g., beads).

In some embodiments, the material and dimensions (e.g., thickness) of a solid support (e.g., a chip) is substantially impermeable to water vapor. In some embodiments, a cover may also be present. In some embodiments, the cover is substantially impermeable to water vapor. For instance, a solid support (e.g., a chip) may include a cover comprising a material known to provide a high vapor barrier, such as metal foil, certain polymers, certain ceramics and combinations thereof. Examples of materials having low water vapor permeability are provided below. In other cases, the material is chosen based at least in part on the shape and/or configuration of the chip. For instance, certain materials can be used to form planar devices whereas other materials are more suitable for forming devices that are curved or irregularly shaped.

A material used to form all or portions of a section or component of any composition described herein may have, for example, a water vapor permeability of less than about 5.0 g·mm/m²·d, less than about 4.0 g·mm/m²·d, less than about 3.0 g·mm/m²·d, less than about 2.0 g·mm/m²·d, less than about 1.0 g·mm/m²·d, less than about 0.5 g·mm/m²·d, less than about 0.3 g·mm/m²·d, less than about 0.1 g·mm/m²·d, or less than about 0.05 g·mm/m²·d. In some cases, the water vapor permeability may be, for example, between about 0.01 g·mm/m²·d and about 2.0 g·mm/m²·d, between about 0.01 g·mm/m²·d and about 1.0 g·mm/m²·d, between about 0.01 g·mm/m²·d and about 0.4 g·mm/m²·d, between about 0.01 g·mm/m²·d and about 0.04 g·mm/m²·d, or between about 0.01 g·mm/m²·d and about 0.1 g·mm/m²·d. The water vapor permeability may be measured at, for example, 40° C. at 90% relative humidity (RH). Combinations of materials with any of the aforementioned water vapor permeabilities may be used in the instant compositions or methods.

In some embodiments, the material and dimensions of a solid support (e.g., a chip) and/or cover may vary. For example, the chip may be configured to provide one or more regions (e.g., liquid containment regions). In certain embodiments, the chip may be configured to provide two or more regions (e.g., liquid containment regions). In certain embodiments, two or more of the regions are fluidically separated from other regions. In one embodiment, all of the regions are fluidically separated from other regions. In some embodiments, all of the regions are fluidically connected. The chip may comprise any number of liquid containment regions. As a non-limiting example, the chip may comprise one, two, three, four, five, six, seven, eight, nine, or ten liquid containment regions, each of which may be fluidically separated from one another. In other embodiments, the chip may comprise one, two, three, four, five, six, seven, eight, nine, or ten liquid containment regions that are fluidically connected to one another.

A solid support (e.g., a chip) described herein may have any suitable volume for carrying out an analysis such as a chemical and/or biological reaction or other process. The entire volume of the solid support may include, for example, any reagent storage areas, analysis regions, liquid containment regions, waste areas, as well as one or more identifiers. In some embodiments, small amounts of reagents and samples are used and the entire volume of the a liquid containment region is, for example, less than or equal to 10 mL, less than or equal to 5 mL, less than or equal to 1 mL, less than or equal to 500 μL, less than or equal to 250 μL, less than or equal to 100 μL, less than or equal to 50 μL, less than or equal to 25 μL, less than or equal to 10 μL, less than or equal to 5 μL, or less than or equal to 1 μL. In some embodiments, small amounts of reagents and samples are used and the entire volume of the a liquid containment region is, for example, at least 10 mL, at least 5 mL, at least 1 mL, at least 500 μL, at least 250 μL, at least 100 μL, at least 50 μL, at least 25 μL, at least 10 μL, at least 5 μL, or at least 1 μL. Combinations of the above-referenced values are also possible.

The length and/or width of the solid support (e.g., chip) may be, for example, less than or equal to 300 mm, less than or equal to 200 mm, less than or equal to 150 mm, less than or equal to 100 mm, less than or equal to 95 mm, less than or equal to 90 mm, less than or equal to 85 mm, less than or equal to 80 mm, less than or equal to 75 mm, less than or equal to 70 mm, less than or equal to 65 mm, less than or equal to 60 mm, less than or equal to 55 mm, less than or equal to 50 mm, less than or equal to 45 mm, less than or equal to 40 mm, less than or equal to 35 mm, less than or equal to 30 mm, less than or equal to 25 mm, or less than or equal to 20 mm. In some embodiments, the length and/or width of the chip may be, for example, at least 300 mm, at least 200 mm, at least 150 mm, at least 100 mm, at least 95 mm, at least 90 mm, at least 85 mm, at least 80 mm, at least 75 mm, at least 70 mm, at least 65 mm, at least 60 mm, at least 55 mm, at least 50 mm, at least 45 mm, at least 40 mm, at least 35 mm, at least 30 mm, at least 25 mm, or at least 20 mm. Combinations of the above-referenced values are also possible. In some embodiments, the thickness of the solid support (e.g., chip) may be, for example, less than or equal to 5 mm, less than or equal to 3 mm, less than or equal to 2 mm, less than or equal to 1 mm, less than or equal to 0.9 mm, less than or equal to 0.8 mm, less than or equal to 0.7 mm, less than or equal to 0.5 mm, less than or equal to 0.4 mm, less than or equal to 0.3 mm, less than or equal to 0.2 mm, or less than or equal to 0.1 mm. In some embodiments, the thickness of the solid support (e.g., chip) may be, for example, at least 5 mm, at least 3 mm, at least 2 mm, at least 1 mm, at least 0.9 mm, at least 0.8 mm, at least 0.7 mm, at least 0.5 mm, at least 0.4 mm, at least 0.3 mm, at least 0.2 mm, or at least 0.1 mm. Combinations of the above-referenced values are also possible. One or more solid supports (e.g., chips) may be analyzed at the same time by any suitable device. An adapter may be used with the one or more solid supports (e.g., chips) in order to insert and securely hold them in the analyzer.

In some embodiments, the solid support (e.g., chip) includes one or more identifiers. Any method or type of identification may be used. For example, an identifier may be, but is not limited to, any type of label such as a bar code or an RFID tag. The identifier may include the name, patient number, social security number, or any other method of identification for a subject. The identifier may also be a randomized identifier of any type useful in a clinical setting.

It should be understood that the solid supports (e.g., chips) and their respective components described herein are exemplary and that other configurations and/or types of solid supports (e.g., chips) and components can be used with the systems and methods described herein.

The binding of a one or more binding partners (e.g., to detect the binding of a protein or other substance of interest including, but not limited to, antigen-bound antibody complexes) may be quantified by any method known in the art. The quantification may, for example, be performed by detection or interrogation of an active molecule bound to an antibody. In a multiplexed format, where more than one assay is being performed on a continuous area, the signals associated with each assay must be differentiable from the other assays. Any suitable strategy known in the art may be used including, but not limited to: (1) using a label with substantially non-overlapping spectral and/or electrochemical properties: (2) using a signal amplification chemistry that remains attached or deposited in close proximity to the tracer itself.

In some embodiments, labeled binding partners (e.g., antibodies or antigen binding fragments) may be used as tracers to detect binding (e.g., using antigen bound antibody complexes). Examples of the types of labels which may be useful for the instant methods and compositions include enzymes, radioisotopes, colloidal metals, fluorescent compounds, magnetic, chemiluminescent compounds, electrochemiluminescent groups, metal nanoparticles, and bioluminescent compounds. Radiolabeled binding partners (e.g., antibodies) may be prepared using any known method and may involve coupling a radioactive isotope such as ¹⁵³Eu, ³H, ³²P, ³⁵S, ⁵⁹Fe, or ¹²⁵I, which can then be detected by gamma counter, scintillation counter or by autoradiography. Binding partners (e.g., antibodies or antigen binding fragments) may alternatively be labeled with enzymes such as yeast alcohol dehydrogenase, horseradish peroxidase, alkaline phosphatase, and the like, then developed and detected spectrophotometrically or visually. The label may be used to react a chromogen into a detectable chromophore (including, for example, if the chromogen is a precipitating dye).

Suitable fluorescent labels may include, but are not limited to: fluorescein, fluorescein isothiocyanate, fluorescamine, rhodamine, Alexa Fluor® dyes (such as Alexa Fluor® 350, Alexa Fluor® 405, Alexa Fluor® 430, Alexa Fluor® 488, Alexa Fluor® 514, Alexa Fluor® 532, Alexa Fluor® 546, Alexa Fluor® 555, Alexa Fluor® 568, Alexa Fluor® 594, Alexa Fluor® 610, Alexa Fluor® 633, Alexa Fluor® 635, Alexa Fluor® 647, Alexa Fluor® 660, Alexa Fluor® 680, Alexa Fluor® 700, Alexa Fluor® 750, or Alexa Fluor® 790), cyanine dyes including, but not limited to: Cy2, Cy3, Cy3.5, Cy5, Cy5.5, Cy7, and Cy7.5, and the like. The labels may also be time- resolved fluorescent (TRF) atoms (e.g., Eu or Sr with appropriate ligands to enhance TRF yield). More than one fluorophore capable of producing a fluorescence resonance energy transfer (FRET) may also be used. Suitable chemiluminescent labels may include, but are not limited to: acridinium esters, luminol, imidazole, oxalate ester, luciferin, and any other similar labels.

Suitable electrochemiluminescent groups for use may include, as a non-limiting example: Ruthenium and similar groups. A metal nanoparticle may also be used as a label. The metal nanoparticle may be used to catalyze a metal enhancement reaction (such as gold colloid for silver enhancement).

Any of the labels described herein or known in the field may be linked to the tracer using covalent or non-covalent means. The label may be presented on or inside an object like a bead (including, for example, a plain bead, hollow bead, or bead with a ferromagnetic core), and the bead is then attached to the binding partner (e.g., an antibody or antigen-binding fragment thereof). The label may also be a nanoparticle including, but not limited to, an up-converting phosphorescent system, nanodot, quantum dot, nanorod, and/or nanowire. The label linked to the antibody may also be a nucleic acid, which might then be amplified (e.g., using PCR) before quantification by one or more of optical, electrical or electrochemical means.

In some embodiments, the binding partner is immobilized on the solid support prior to formation of binding complexes. In other embodiments, immobilization of the antibody and antigen-binding fragment is performed after formation of binding complexes.

In one embodiment, immunoassay methods disclosed herein comprise immobilizing binding partners (e.g., antibodies or antigen-binding fragments) to a solid support (e.g., a chip); applying a sample (e.g., an endometrial fluid sample) to the solid support under conditions that permit binding of the expression product of a biomarker (e.g., a protein) to one or more binding partners (e.g., one or more antibodies or antigen-binding fragments), if present in the sample; removing the excess sample from the solid support; detecting the bound complex (using, e.g., detectably labeled antibodies or antigen-binding fragments) under conditions that permit binding (e.g., of an expression product to the antigen-bound immobilized antibodies or antigen-binding fragments); washing the solid support and assaying for the label.

Reagents can be stored in or on a chip for various amounts of time. For example, a reagent may be stored for longer than 1 hour, longer than 6 hours, longer than 12 hours, longer than 1 day, longer than 1 week, longer than 1 month, longer than 3 months, longer than 6 months, longer than 1 year, or longer than 2 years. Optionally, the chip may be treated in a suitable manner in order to prolong storage. For instance, chips having stored reagents contained therein may be vacuum sealed, stored in a dark environment, and/or stored at low temperatures (e.g., below 4° C. or 0° C.). The length of storage depends on one or more factors such as the particular reagents used, the form of the stored reagents (e.g., wet or dry), the dimensions and materials used to form the substrate and cover layer(s), the method of adhering the substrate and cover layer(s), and how the chip is treated or stored as a whole. Storing of a reagent (e.g., a liquid or dry reagent) on a solid support material may involve covering and/or sealing the chip prior to use or during packaging.

Any solid state assay device described herein may be included in a kit. The kit may include any packaging useful for such devices. The kit may include instructions for use in any format or language. The kit may also direct the user to obtain further instructions from one or more locations (physical or electronic). The included instructions can comprise a description of how to use the components contained in the kit for measuring the level of a biomarker set (e.g., protein biomarker or nucleic acid biomarker) in a biological sample collected from a subject, such as a human patient. The instructions relating to the use of the kit generally include information as to the amount of each component and suitable conditions for performing the assay methods described herein.

The components in the kits may be in unit doses, bulk packages (e.g., multi-dose packages), or sub-unit doses. The kit can also comprise one or more buffers as described herein but not limited to a coating buffer, a blocking buffer, a wash buffer, and/or a stopping buffer.

The kits of this present disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an PCR machine, a nucleic acid array, or a flow cytometry system.

Kits may optionally provide additional components such as interpretive information, such as a control and/or standard or reference sample. Normally, the kit comprises a container and a label or package insert(s) on or associated with the container. In some embodiments, the present disclosure provides articles of manufacture comprising contents of the kits described above.

### EXAMPLES

### Materials and Methods

A total of 13 LM and 13 LMS from formalin-fixed paraffin-embedded (FFPE) samples were collected, processed and histologically confirmed according to World Health Organization criteria.^{35,36} Of note, one of the initially diagnosed LMS was confirmed as inflammatory myofibroblastic tumor (IMT, sample IMT01) after the molecular analysis and subsequent histological validation. The research was approved by the Institutional Review Board of University Hospital La Fe (2016/0118).

Illumina TruSight Tumor 170 kit was used for targeted sequencing of DNA and RNA coding regions for solid tumor-associated genes. Bioinformatic analysis for small variants, including point mutations and indels, was performed by Pisces³⁷. CNVs were detected by CRAFT. Additionally, RNA Splice Variant Caller software was used for splice variant calling and differential expression analysis was performed using the edgeR package³⁸ from Bioconductor software.³⁹ Lastly, fusion genes were identified with Manta RNA Fusion Calling software and validated by IHC and FISH.

### Example 1—Patient Characteristics

Patients with LM diagnosis had a median age of 43 years (range: 30-48 years), while LMS patients 55 (range: 44-67 years). All tumors were collected during primary resection, and 50% of LMS tumors were high-grade. Tumor size varied from 12-150 mm (median 71.6±9.4 mm) in LM and 80-230 mm (median 160±32.9 mm) in LMS (Table 1). Histological information estimated ˜69% of necrosis in LMS samples and ˜78% with high mitotic activity (Table 2).

TABLE 1
Clinical and pathological features of patients diagnosed with leiomyoma and leiomyosarcoma.

| | | | | | | | | | | FIGO |
|---|---|---|---|---|---|---|---|---|---|---|
| | | | | | | | | | Tumor | Staging |
| Tumor | Case | | | | | Miscar- | Clinical | Surgical | size | Classi- |
| type | ID | Source | Age | Ethnicity | Parity | riage | History | procedure | (mm) | fication |
| LM | 16LM | La Fe | 39 | Caucasian | No | No | N/A | Laparotomic | 60 | 4 |
| | | | | | | | | Myomectomy | | |
| | 17LM | La Fe | 47 | Caucasian | Yes | Yes | Sterility | Laparotomic | 65 | 5 |
| | | | | | | | | Myomectomy | | |
| | 22LM | La Fe | 47 | Caucasian | Yes | No | Family | Laparoscopic | 68 | 4 |
| | | | | | | | history | hysterectomy | | |
| | 23LM | La Fe | 47 | Caucasian | Yes | Yes | Family | Laparoscopic | 60 | 5 |
| | | | | | | | history | hysterectomy | | |
| | 25LM | La Fe | 47 | Caucasian | Yes | Yes | Family | Laparoscopic | 110 | 6 |
| | | | | | | | history | hysterectomy | | |
| | 28LM | La Fe | 39 | Caucasian | No | No | Family | Laparotomic | 82 | 7 |
| | | | | | | | history | Myomectomy | | |
| | 30LM | La Fe | 46 | Caucasian | Yes | Yes | Family | Laparoscopic | 86 | 5 |
| | | | | | | | history | hysterectomy | | |
| | 32LM | La Fe | 45 | Caucasian | Yes | Yes | N/A | Laparoscopic | 12 | 4 |
| | | | | | | | | hysterectomy | | |
| | 33LM | La Fe | 30 | Caucasian | No | No | N/A | Laparotomic | 90 | 2-5 |
| | | | | | | | | Myomectomy | | |
| | 34LM | La Fe | 40 | Caucasian | No | No | N/A | Laparotomic | 150 | 8 |
| | | | | | | | | hysterectomy | | |
| | 35LM | La Fe | 48 | Caucasian | Yes | Yes | Family | Laparotomic | 35 | 4 |
| | | | | | | | history | hysterectomy | | |
| | 36LM | La Fe | 48 | Caucasian | Yes | No | N/A | Laparoscopic | 70 | 4 |
| | | | | | | | | hysterectomy | | |
| | 41LM | La Fe | 44 | Caucasian | Yes | No | N/A | Laparoscopic | 24 | 2-5 |
| | | | | | | | | hysterectomy | | |
| LMS | LMS02 | La Fe | 50 | Latin | Yes | Yes | N/A | Laparotomic | 90 | IB |
| | | | | | | | | hysterectomy | | |
| | LMS03 | La Fe | 64 | Caucasian | N/A | N/A | N/A | Laparotomic | 230 | IV |
| | | | | | | | | hysterectomy | | |
| | LMS04 | La Fe | 53 | Caucasian | Yes | No | N/A | Laparotomic | 120 | IV |
| | | | | | | | | hysterectomy | | |
| | LMS05 | La Fe | 55 | Caucasian | Yes | No | Family | Laparotomic | 200 | IIIB |
| | | | | | | | history | hysterectomy | | |
| | LMS06 | Origene | 39 | Asian | N/A | N/A | N/A | N/A | N/A | N/A |
| | LMS08 | Origene | 50 | Caucasian | N/A | N/A | N/A | N/A | N/A | IIB |
| | LMS09 | Origene | 54 | Caucasian | N/A | N/A | N/A | N/A | N/A | IC |
| | LMS10 | Origene | 55 | Caucasian | N/A | N/A | N/A | N/A | N/A | IIB |
| | LMS11 | Origene | 55 | Caucasian | N/A | N/A | N/A | N/A | N/A | IC |
| | LMS12 | Origene | 56 | Caucasian | N/A | N/A | N/A | N/A | N/A | IIIB |
| | LMS13 | Origene | 60 | N/A | N/A | N/A | N/A | N/A | N/A | N/A |
| | LMS14 | Origene | 62 | N/A | N/A | N/A | N/A | N/A | N/A | N/A |
| | LMS15 | Origene | 67 | Caucasian | N/A | N/A | N/A | N/A | N/A | IIIA |
| IMT | IMT01* | La Fe | 60 | Caucasian | Yes | No | N/A | Laparoscopic | 80 | IB |
| | | | | | | | | hysterectomy | | |

*Initially diagnosed as LMS and subsequently confirmed as inflamatory myofibroblastic tumor (IMT).

TABLE 2
Morphological and histological characteristics
of leiomyosarcoma tumors and patient follow-up.

| | Tumor | | | | Hystological | Outcome |
|---|---|---|---|---|---|---|
| Case ID | differentiation | Necrosis | Mitotic activity | Atypia | Variant | follow up |
| IMT01* | N/A | Present | >19/10 HPF | Moderate | N/A | Alive |
| LMS02 | Poorly | Present | >19/10 HPF | Severe | Myxoid | Alive |
| | differentiated | | | | | |
| LMS03 | Moderated | Present | 1-9/10 HPF | Moderate | Spindle cell | Deceased |
| | differentiated | | | | | |
| LMS04 | Poorly | Present | >19/10 HPF | Moderate | Spindle cell | Deceased |
| | differentiated | | | | | |
| LMS05 | Poorly | Present | >19/10 HPF | Severe | Spindle cell | Deceased |
| | differentiated | | | | | |
| LMS06 | Well | Absent | >10/10 HPF | Moderate/severe | Spindle cell | N/A |
| | differentiated | | | | | |
| LMS08 | N/A | Absent | N/A | N/A | N/A | N/A |
| LMS09 | Poorly | Absent | >40/10 HPF | Moderate | N/A | N/A |
| | differentiated | | | | | |
| LMS10 | Poorly | Present | >20/10 HPF | Moderate/severe | Spindle cell | N/A |
| | differentiated | | | | | |
| LMS11 | Well | Absent | <10/10 HPF | Moderate | N/A | Alive |
| | differentiated | | | | | |
| LMS12 | N/A | Absent | N/A | N/A | N/A | N/A |
| LMS13 | Well | Present | >10/10 HPF | N/A | N/A | N/A |
| | differentiated | | | | | |
| LMS14 | N/A | Present | N/A | N/A | N/A | N/A |
| LMS15 | N/A | Present | N/A | N/A | Myxoid | N/A |

*Initially diagnosed as LMS and subsequently confirmed as inflamatory myofibroblastic tumor (IMT).

### Example 2—Comparative Genomic Analysis of Leiomyoma and Leiomyosarcoma

A comparative screen for somatic mutations between LM and LMS samples was conducted. Average coverage reached a mean depth of 3535x, with a minimum coverage of 6 reads. An average of 20 mutations in 82 genes in LM and 22 mutations in 105 genes in LMS samples were observed (Table 3). The LM group represented ˜3% of deletions, ˜9% of insertions, and ˜88% of SNPs, while in LMS ˜5% were deletions, ˜9% insertions, and ˜86% SNPs. Regarding IMT01, 10 mutations in 8 genes were observed including ˜10% of deletions and ˜90% of SNPs (Table 3).

TABLE 3
Affected genes and actionable mutations in leiomyoma and leiomyosarcoma groups.

| | Variants | | | | | | |
|---|---|---|---|---|---|---|---|
| | per | | | | | | |
| Tumor | sample | Genes | | Samples | | | |
| type | (mean) | (n) | Gene description | (n) | DELs | INSs | SNP |
| LM | 20 | 82 | FGFR2, KLLN, PTEN, ATM, KMT2A, MTOR, NRAS, | 13 | 5 | 19 | 175 |
| | | | NOTCH2, FGF19, AP001888.1, FGF3, MRE11A, | | (2.51%) | (9.55%) | (87.94%) |
| | | | MDM4, PTPN11, SDCCAG8, FGF6, ERBB3, MDM2, | | | | |
| | | | NA, LAMP1, FGF9, FLT1, BRCA2, MYCL, | | | | |
| | | | RP11-982M15.2, MPL, HPDL, SLC35F4, | | | | |
| | | | RAD51B, RAD51, IDH2, TSC2, SLX4, CREBBP, | | | | |
| | | | RAD51L3-RFFL, TP53, RBFOX3, STK11, NOTCH3, | | | | |
| | | | TGFBR3, AKT2, GNAS-AS1, ERG, MYCNOS, BARD1, | | | | |
| | | | EP300, DNMT3A, MSH2, MSH6, VHL, RAF1, PIK3CB, | | | | |
| | | | PIK3CA, TFRC, MLH1, BAP1, TET2, FGFR3, | | | | |
| | | | PDGFRA, MRPS18C, APC, HMGXB3, CSF1R, PDGFRB, | | | | |
| | | | FGFR4, FGF10, ESR1, BYSL, CCND3, SMO, DPP6, | | | | |
| | | | EGFR, CDK6, MYC, NRG1, NOTCH1, MLLT3, | | | | |
| | | | RP11-145E5.5, JAK2, GNAQ, PTCH1, AR | | | | |
| LMS | 22 | 105 | FGF8, RET, PTEN, ATM, CADM1, KMT2A, NOTCH2, | 13 | 12 | 20 | 199 |
| | | | MCL1, DDR2, CCND1, FGF19, FGF3, MDM4, KRAS, | | (5.19%) | (8.66%) | (86.15%) |
| | | | SDCCAG8, CCND2, RP11-611O2.2, MDM2, ARID1A, | | | | |
| | | | FGF14, LAMP1, NA, FGF9, FLT1, ALOX5AP, | | | | |
| | | | BRCA2, RB1, MYCL, MPL, HPDL, MUTYH, | | | | |
| | | | RAD54L, RAD51B, FANCI, TSC2, PALB2, NLRC3, | | | | |
| | | | SLX4, CREBBP, CDH1, RP11-525K10.1, | | | | |
| | | | RAP1GAP2, RAD51L3-RFFL, ERBB2, BRCA1, TEX14, | | | | |
| | | | RPS6KB1, TP53, RBFOX3, BCL2, STK11, NOTCH3, | | | | |
| | | | JAK3, TGFBR3, CCNE1, AKT2, ERCC2, PPP1R13L, | | | | |
| | | | PIK3CD, GNAS, ERG, ERBB4, BARD1, RNA5SP495, | | | | |
| | | | CHEK2, RP1-302D9.3, EP300, RNU6-688P, MSH6, | | | | |
| | | | VHL, MKRN2, ATR, MLH1,, TET2, FGF2, FGFR3, | | | | |
| | | | PDGFRA, KDR, FGF5, APC, HMGXB3, CSF1R, | | | | |
| | | | PDGFRB, FGF10, PIK3R1, DHFR, ROS1, HIVEP1, | | | | |
| | | | ESR1, BYSL, MET, SMO, BRAF, DPP6, CARD11, | | | | |
| | | | EGFR, CASC11.NRG1, FGFR1, NOTCH1, MLLT3, | | | | |
| | | | LINGO2, PTCH1, AR | | | | |
| IMT | 10 | 8 | FGF19, FGF3, CCNE1, BARD1, FGFR3, FGF5, | 1 | 1 | | 9 |
| | | | PDGFRB, SMO | | (10%) | | (90%) |

Next, specific variants in at least two LM or LMS tumors were focused on. The most frequently affected variants in LM were FGF6 and MRE11A, affecting 4 and 2 samples respectively. In LMS, RET and FGF5 were the most common altered genes, affecting 3 samples (Table 4).

TABLE 4
Unique variants in leiomyoma (LM) and leiomyosarcoma (LMS) samples

| Tumor | | Number of | | | |
|---|---|---|---|---|---|
| type | Variant_id | samples | Sample description | Transcript | Consequence |
| LM | chr12-4551244-T-G | 4 | 22LM, 30LM, 32LM, 35LM | ENST0000022883 7 | intron_variant |
| | chr11-94192599-G-T | 2 | 16LM, 17LM | ENST0000032392 9 | missense_variant |
| LMS | chr10-43597827-C-A | 3 | LMS12, LMS14, LMS15 | ENST0000035571 0 | synonymous_variant |
| | chr4-81206898-TAA-T | 3 | , LMS04 LMS12, LMS13 | ENST0000031246 5 | intron_variant |

| Tumor | | | | | |
|---|---|---|---|---|---|
| type | HGVSc | HGVSp | HGNC | Exons | COSMICID |
| LM | ENST00000228837.2:c.450+2055A > C | — | FGF6 | — | — |
| | ENST00000323929.3:c.1475C > A | ENSP00000325863.3:p.Ala492Asp | MRE11A | 13/20 | — |
| LMS | ENST00000355710.3:c.375C > A | ENST00000355710.3:c.375C > A(p.=) | RET | 3/20 | COSM502115 3 |
| | | | | | COSM502115 4 |
| | ENST00000312465.7:c.460-579_460-578delAA | — | FGF5 | — | — |

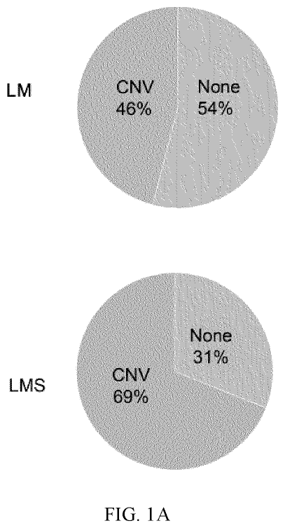

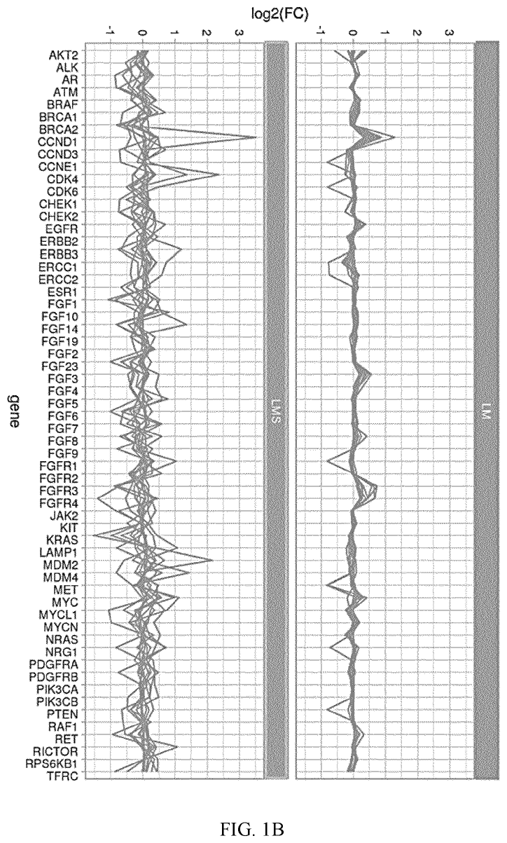

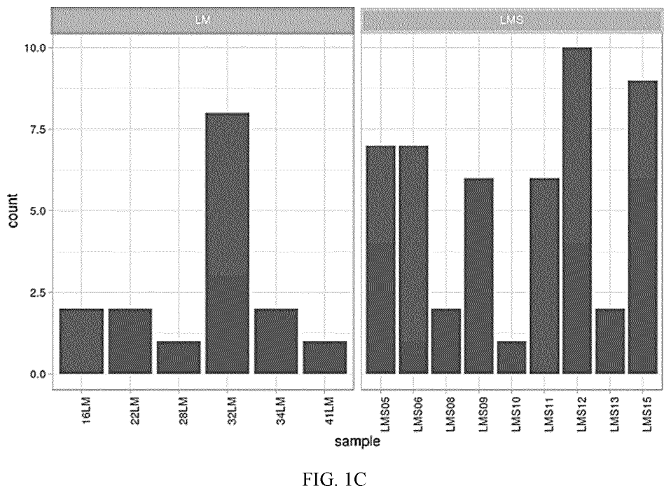

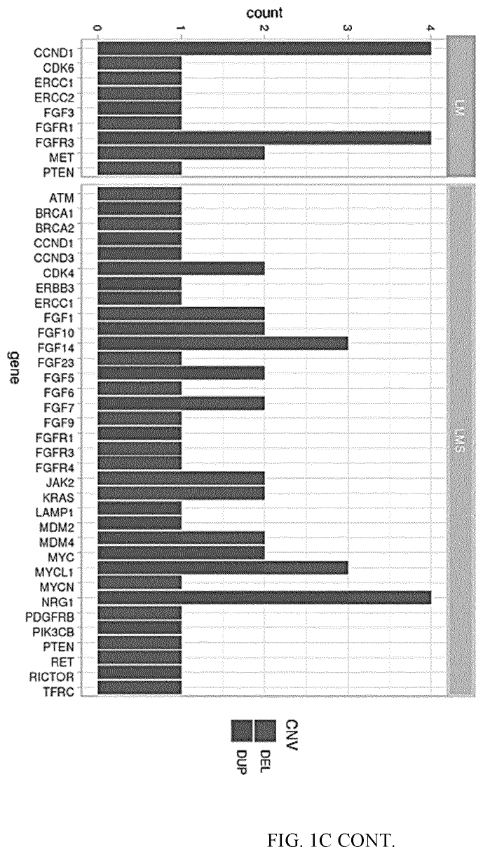

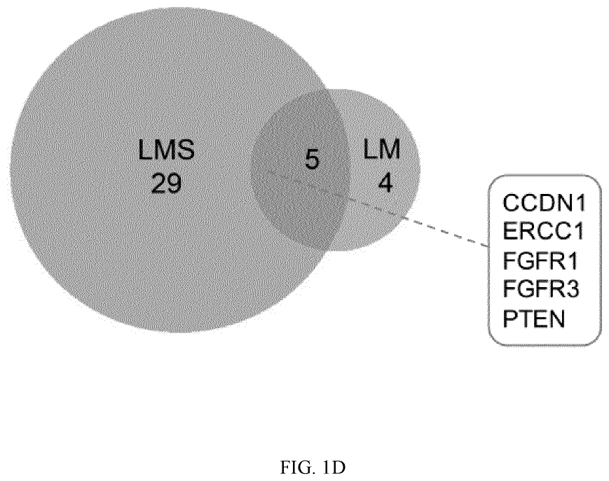

Comparative analysis of CNVs showed that there were more CNVs in LMS (69%) compared to LM (46%) cases (FIG. 1A; Table 5). Interestingly, when their distribution was represented by specimen group and gene, the highest heterogenicity was observed in LMS, with more deletions and duplications than LM (FIGS. 1B, 1C). Pairwise comparisons showed significant differences (p≤0.05).

TABLE 5
Small variants and copy number variants in leiomyoma (LM) and leiomyosarcoma (LMS) samples.

| | | Small Variants | | | | | | |
|---|---|---|---|---|---|---|---|---|
| | | Total | | | | Copy Number Variants (CNVs) | | |
| Tumor | | Small | | | | Total | | |
| type | CaseID | Variants | SNVs | DELs | INSs | CNVs | Deletions | Duplications |
| LM | 16LM | 20 | 17(85%) | 1(5%) | 2(10%) | 2 | 2(100%) | 0(0%) |
| | 17LM | 16 | 15(93.75%) | 0(0%) | 1(6.25%) | 0 | 0(0%) | 0(0%) |
| | 22LM | 12 | 11(91.67%) | 0(0%) | 1(8.33%) | 2 | 0(0%) | 2(100%) |
| | 23LM | 13 | 11(84.62%) | 0(0%) | 2(15.38%) | 0 | 0(0%) | 0(0%) |
| | 25LM | 37 | 33(89.19%) | 1(2.7%) | 3(8.11%) | 0 | 0(0%) | 0(0%) |
| | 28LM | 22 | 21(95.45%) | 0(0%) | 1(4.55%) | 1 | 0(0%) | 1(100%) |
| | 30LM | 17 | 15(88.24%) | 0(0%) | 2(11.76%) | 0 | 0(0%) | 0(0%) |
| | 32LM | 15 | 13(86.67%) | 1(6.67%) | 1(6.67%) | 8 | 5(62.5%) | 3(37.5%) |
| | 33LM | 2 | 2(100%) | 0(0%) | 0(0%) | 0 | 0(0%) | 0(0%) |
| | 34LM | 11 | 9(81.82%) | 1(9.09%) | 1(9.09%) | 2 | 0(0%) | 2(100%) |
| | 35LM | 18 | 15(83.33%) | 1(5.56%) | 2(11.11%) | 0 | 0(0%) | 0(0%) |
| | 36LM | 15 | 12(80%) | 0(0%) | 3(20%) | 0 | 0(0%) | 0(0%) |
| | 41LM | 1 | 1(100%) | 0(0%) | 0(0%) | 1 | 0(0%) | 1(100%) |
| LMS | LMS02 | 41 | 37(90.24%) | 2(4.88%) | 2(4.88%) | 0 | 0(0%) | 0(0%) |
| | LMS03 | 19 | 18(94.74%) | 0(0%) | 1(5.26%) | 0 | 0(0%) | 0(0%) |
| | LMS04 | 25 | 21(84%) | 2(8%) | 2(8%) | 0 | 0(0%) | 0(0%) |
| | LMS05 | 15 | 14(93.33%) | 0(0%) | 1(6.67%) | 7 | 3(42.8%) | 4(57.2%) |
| | LMS06 | 21 | 20(95.24%) | 0(0%) | 1(4.76%) | 7 | 6(85.7%) | 1(14.3%) |
| | LMS08 | 12 | 9(75%) | 1(8.33%) | 2(16.67%) | 2 | 0(0%) | 2(100%) |
| | LMS09 | 13 | 11(84.62%) | 2(15.38%) | 0(0%) | 6 | 0(0%) | 6(100%) |
| | LMS10 | 10 | 7(70%) | 0(0%) | 3(30%) | 1 | 0(0%) | 1(100%) |
| | LMS11 | 10 | 10(100%) | 0(0%) | 0(0%) | 6 | 6(100%) | 0(0%) |
| | LMS12 | 13 | 10(76.92%) | 1(7.69%) | 2(15.38%) | 10 | 6(60%) | 4(40%) |
| | LMS13 | 17 | 13(76.47%) | 2(11.76%) | 2(11.76%) | 2 | 0(0%) | 2(100%) |
| | LMS14 | 27 | 23(85.19%) | 1(3.7%) | 3(11.11%) | 0 | 0(0%) | 0(0%) |
| | LMS15 | 8 | 6(75%) | 1(12.5%) | 1(12.5%) | 9 | 3(33.3%) | 6(66.7%) |
| IMT | IMT01* | 10 | 9(90%) | 1(10%) | 0(0%) | 8 | 6(75%) | 2(25%) |

*Initially diagnosed as LMS and subsequently confirmed as inflamatory myofibroblastic tumor (IMT).

The most frequent duplications in LM were on chromosomes 11 and 4, affecting CCND1 and FGFR3, while the most frequent deletion was detected on chromosome 7, affecting MET. For LMS samples, chromosomes 5, 9, and 12, were the most affected, with deletions encompassing FGF1, JAK2 and KRAS. The most frequently amplified genes in LMS were CDK4, FGF10, FGF5 and MYC on chromosome 12, 5, 4 and 8, respectively. Duplications and deletions in the LMS group were in FGF14, FGF7, MDM4, MYCL1, and NRG1 (FIG. 1C; Table 6). For IMT sample, six deletions affecting CCND3, ERBB3, FGF7, JAK2, NRAS, RAF1 and two duplications including FGF10 and FGFR4 were found.

TABLE 6
Relevant CNVs in leiomyoma and leiomyosarcoma samples

| | | | | Freq. | Total | |
|---|---|---|---|---|---|---|
| Tumor | | | | Group | Freq | Supporting |
| type | Genes | Type | Size | (%) | (%) | studies |
| LM | CCND1 | Dup | 13236 | 30.8 | 14.8 | Musgrove et al., |
| | | | | | | 2011 |
| | FGFR3 | Dup | 13328 | 30.8 | 14.8 | Yu et al., 2008 |
| | MET | Del | 97040 | 15.4 | 7.4 | Scherer. 1997; |
| | | | | | | Toro et al., 2003 |
| LMS | FGF1 | Del | 102108 | 13.3 | 7.1 | Zhou et al., 2016 |
| | JAK2 | Del | 104804 | 20 | 10.7 | Hayashi et al., |
| | | | | | | 2008 |
| | KRAS | Del | 38360 | 13.3 | 7.1 | Schachtschneider |
| | | | | | | et al. 2017 |
| | CDK4 | Dup | 3482 | 13.3 | 7.1 | Francis et al., |
| | | | | | | 2017 |
| | FGF10 | Dup | 83688 | 20 | 10.7 | Zhou et al., 2016 |
| | FGF5 | Dup | 23615 | 13.3 | 7.1 | Zhan et al.. 1988; |
| | | | | | | Giacominiet al., |
| | | | | | | 2016 |
| | MYC | Dup | 4364 | 13.3 | 7.1 | Jeffers et al., |
| | | | | | | 1995 |
| | FGF14 | Del/ | 678848 | 20 | 10.7 | Presta et al., 2017 |
| | | Dup | | | | |
| | FGF7 | Del/ | 40556 | 20 | 10.7 | Zhou et al., 2016 |
| | | Dup | | | | |
| | MDM4 | Del/ | 39564 | 13.3 | 7.1 | Toledo and |
| | | Dup | | | | Wahl., 2007; |
| | | | | | | Atwal et al., |
| | | | | | | 2009; |
| | MYCL1 | Del/ | 4517 | 20 | 10.7 | Barnabas et al., |
| | | Dup | | | | 2014; Groisberg |
| | | | | | | et al., 2017 |
| | NRG1 | Del/ | 1124420 | 26.7 | 14.3 | Yatsenko et al., |
| | | Dup | | | | 2017 |

Genetic modifications were shared between LM and LMS in CCDN1, ERCC1, FGFR1, FGFR3, and PTEN. While ERCC1 and FGFR1 were affected by deletions in LM, these genes were duplicated in LMS. Conversely, FGFR3 presented duplications in LM and deletions in LMS, while no differences between LM and LMS were detected for CCND1 and PTEN. Finally, 4 CNVs were found in LM, while 29 were exclusively present in LMS (FIG. 1D).

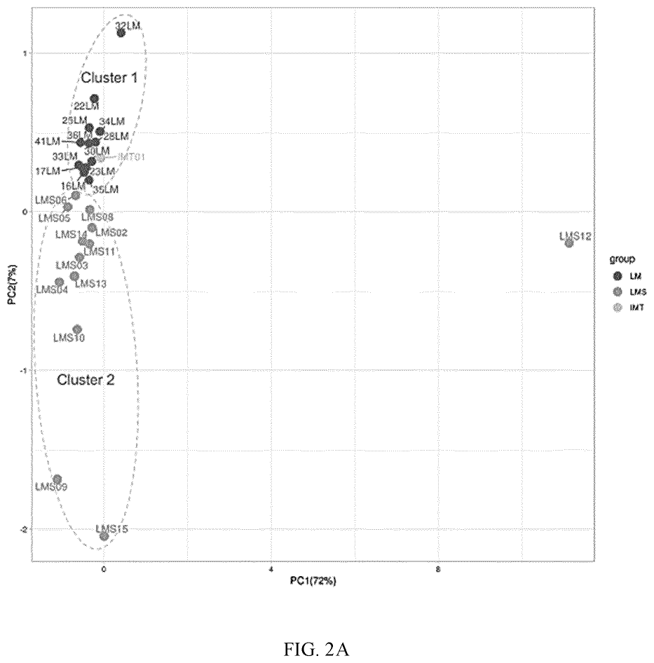

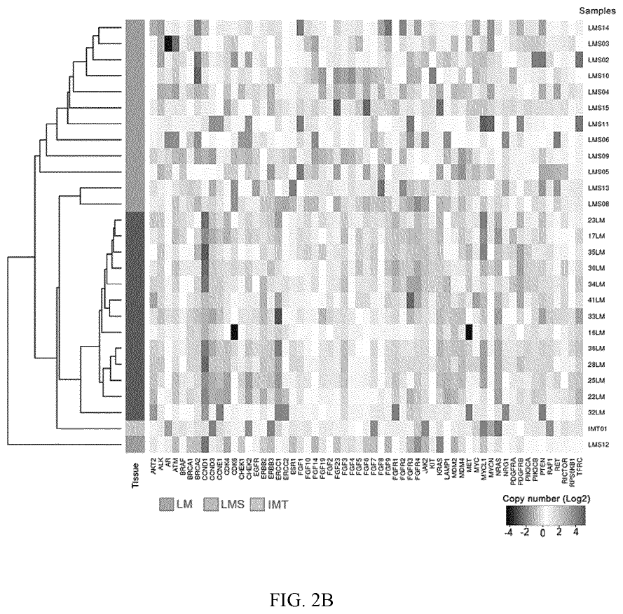

Principal component analysis (PCA) demonstrated that LM and LMS samples clustered separately according to tissue of origin except for LMS12, which was considered an outlier (FIG. 2A). Interestingly, IMT01, initially diagnosed as LMS, was grouped among LM samples, suggesting an additional molecular subtype. Unsupervised hierarchical clustering analysis recreated the PCA clustering structure (FIG. 2B). As previously observed, LM specimens were grouped in a homogeneous cluster encompassing thirteen samples, while LMS samples were more heterogeneous. Specifically, one main cluster was observed including ten LMS samples, another two samples (LMS08 and LMS13) clustered separately and LMS12 which was considered an outlier characterized by distinctive alterations in CCDN1 (FIG. 2B). To note, IMT sample showing specific alterations in AKT2, ALK and FGF7 clustered separately from LM and LMS group, supporting a different molecular subtype.

Preferred sets of biomarkers for Leiomyoma (LM) and Leiomyosarcoma (LMS) are represented in Tables 7 and 8, respectively.

TABLE 7
Leiomyoma variants: results based on FIG. 1C:

| | Biomarkers Indicative of |
|---|---|
| Genetic Signatures | Leiomyoma (LM) |
| CNV Duplication (same as | FGFR3 |
| Amplification) | |
| CNV Deletion | MET |

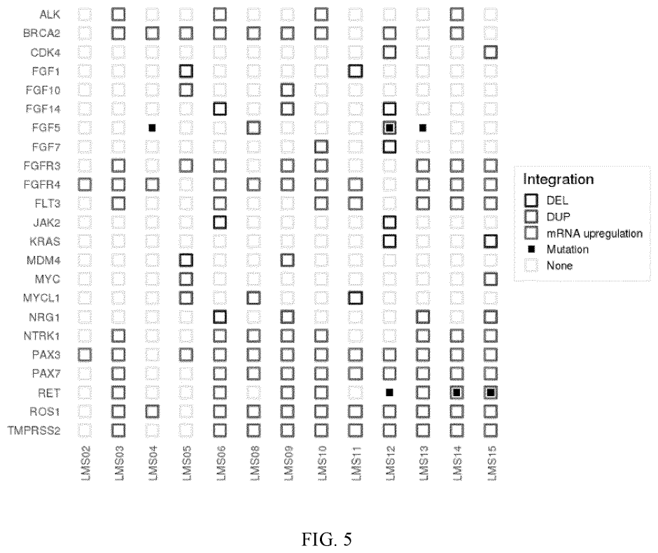

TABLE 8
Leiomyosarcoma variants: results based on FIG. 1C and FIG. 5:

| | Biomarkers Indicative of |
|---|---|
| Genetic Signatures | Leiomyosarcoma (LMS) |
| CNV Duplication (same as | CDK4, FGF10, FGF5, MYC, MYCL1, |
| Amplification) | NRG1 |
| CNV Deletion | FGF1, FGF14, JAK2, KRAS |
| CNV Duplication & Deletion | FGF14, FGF7, MDM4, MYCL1, NRG1 |
| SNV | FGF5, RET |
| mRNA upregulation | ALK, BRCA2, FGFR3, FGFR4, FLT3, |
| | NTRK1, PAX3, PAX7, RET, ROS1, |
| | TMPRSS2 |

### Example 3—Differentially Expressed Genes in Leiomyoma and Leiomyosarcoma

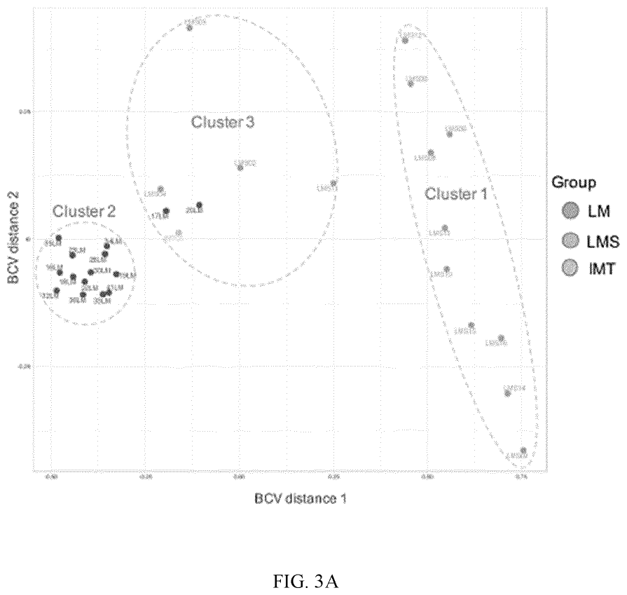

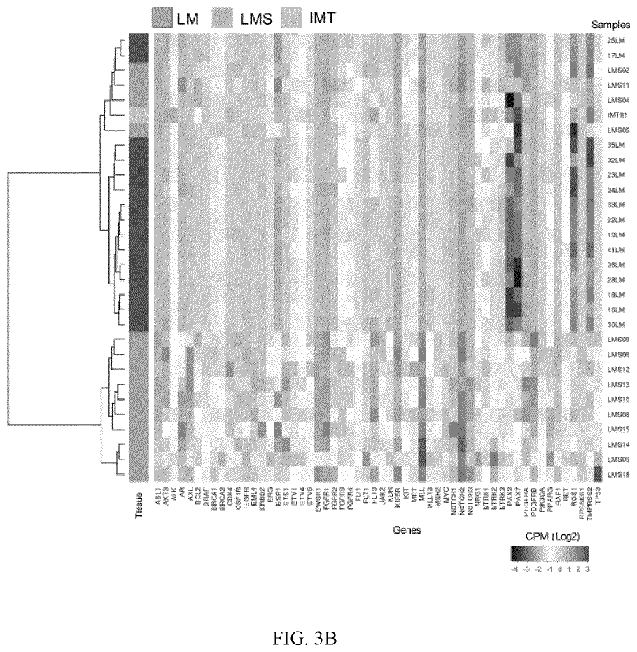

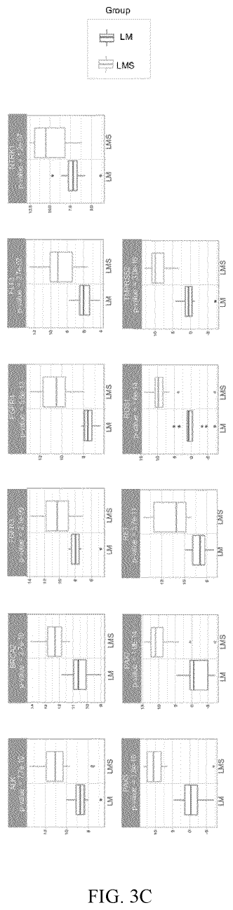

Transcriptome sequencing results identified 3 groups: a homogeneous group with LMS samples (cluster 1), a homogeneous group composed by LM (cluster 2) and a heterogeneous group composed by LM, LMS and IMT samples (Cluster 3; FIG. 3A). Unsupervised hierarchical clustering also categorized 3 expression clusters. In cluster 1, LMS samples were together into the same group, cluster 2 corresponded with a homogeneous group including LM samples and cluster 3 included some of LMS samples, the IMT specimen and two LM samples (17LM and 25LM), supporting previous results (FIG. 3B).

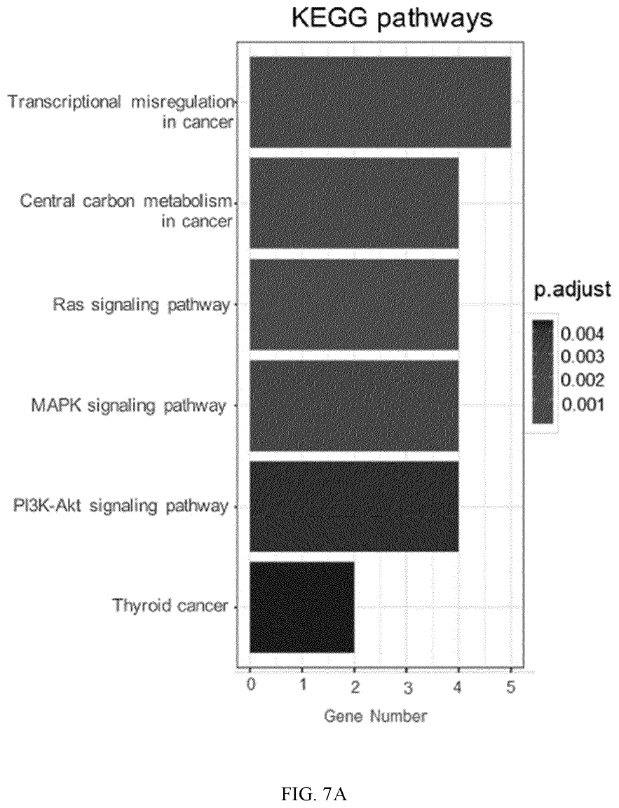

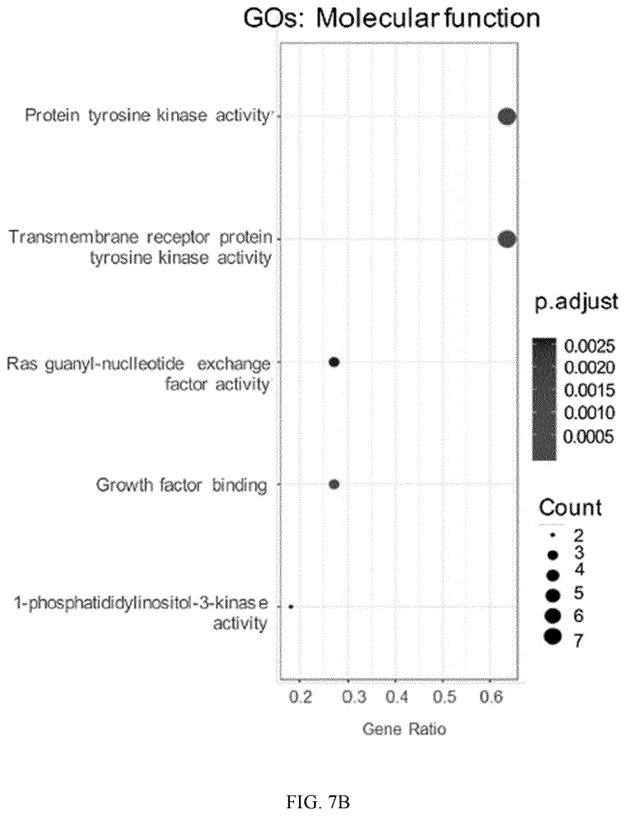

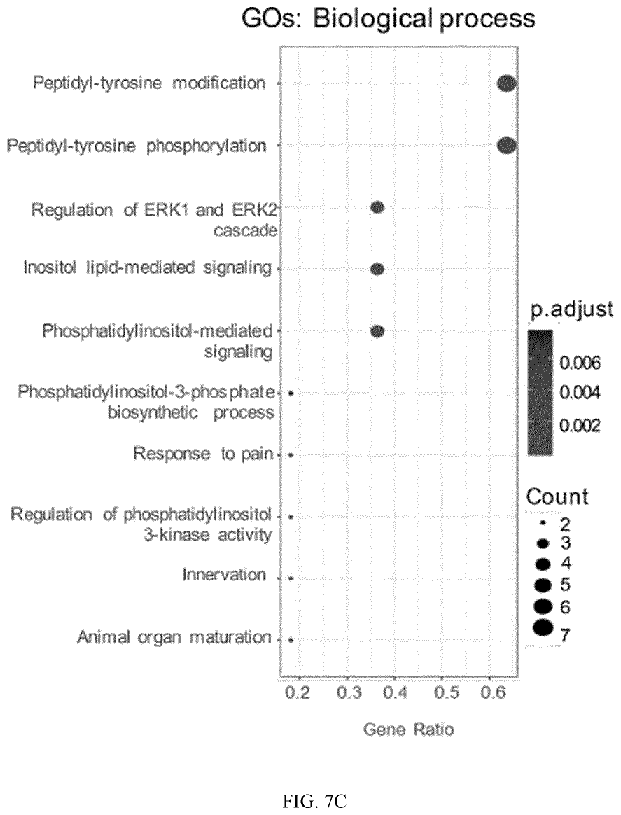

Next, targetable differential expression was identified in LMS and LM. Overall, 11 of 55 genes—ALK, BRCA2, FGFR3, FGFR4, FLT3,NTRK1, PAX3, PAX7, RET, ROS1, and TMPRSS2—were significantly upregulated in LMS compared to LM (p≤0.0.5) (FIG. 3C; Table 9). These differentially expressed genes were then evaluated for molecular functions and biological processes, considering only pathways with at least 2 annotated genes. KEGG database analysis of implicated functions revealed an overrepresentation of pathways involved in transcriptional misregulation and central carbon metabolism in cancer as well as RAS/MAPK and PI3K-AKT signaling pathways and thyroid cancer (p≤0.05; FIG. 7A). Moreover, Gene Ontology (GO) enrichment analysis showed protein tyrosine kinase activity as a main molecular function involved in the tumorigenic process (FIG. 7B), as well as peptidyl-tyrosine modification/phosphorylation as a principal biological process (FIG. 7C).

TABLE 9
Most differentially expressed genes in leiomyosarcoma.

| Gene | logFC | logCPM | LR | P-Value |
|---|---|---|---|---|
| ALK | 2.73 | 11.87 | 37.82 | 7.74E−10 |
| BRCA2 | 2.03 | 11.98 | 39.86 | 2.72E−10 |
| FGFR3 | 3.41 | 10.51 | 35.86 | 2.12E−09 |
| FGFR4 | 3.66 | 10.33 | 51.92 | 5.77E−13 |
| FLT3 | 3.92 | 9.17 | 26.46 | 2.68E−07 |
| NTRK1 | 3.32 | 10.05 | 28.03 | 1.19E−07 |
| PAX3 | 9.67 | 10.44 | 63.27 | 1.8E−15 |
| PAX7 | 10.37 | 10.95 | 58.60 | 1.93E−14 |
| RET | 3.07 | 11.05 | 42.96 | 5.56E−11 |
| ROS1 | 8.96 | 10.16 | 58.99 | 1.58E−14 |
| TMPRSS2 | 8.99 | 9.56 | 66.81 | 2.98E−16 |

### Example 4—Novel ALK Receptor Tyrosine Kinase-Tensin 1 fusion

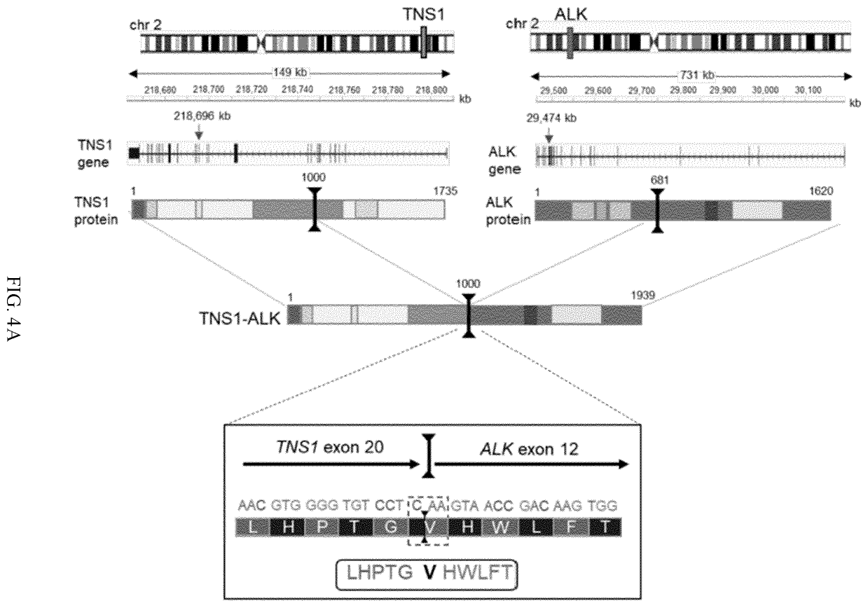

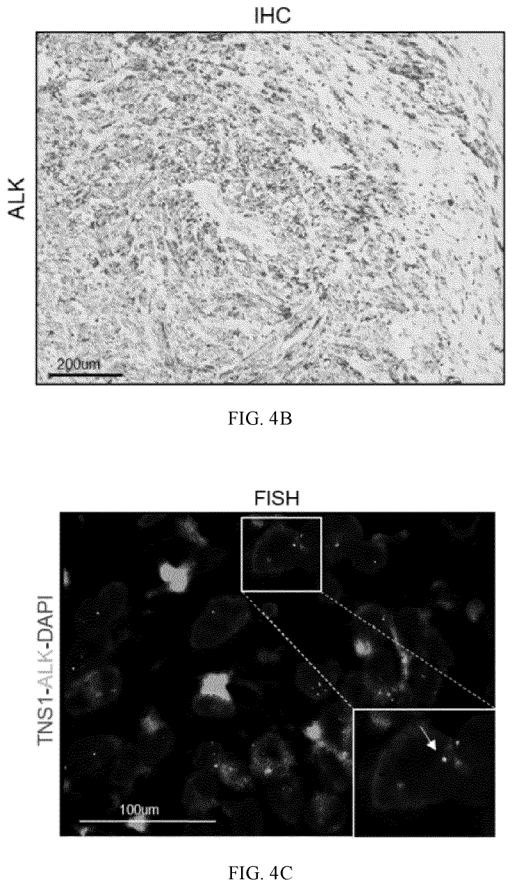

While RNA-seq was performed using paired-end sequencing, fusion transcripts could be detected from 55 genes targeted by the TST170 panel, meeting a minimum threshold score of ≥0.98. One sample, IMT01, initially diagnosed as LMS01, showed an ALK Receptor Tyrosine Kinase—Tensin 1 (TNS1) fusion (FIG. 4A). IHC and FISH were used to validate the ALK rearrangement.^{40,41} As shown, IHC (FIG. 4B) demonstrated diffused strong ALK-positive staining that was confirmed by FISH to be an ALK translocation (FIG. 4C).

### Example 5—Integrative Differential Profile for Specific Pathways and Targetable Mutations

Integration of all results from CNVs, small variants, and gene expression data revealed 5 genes—FGFR4, PAX3, PAX7, ROS1 and TMPRSS2—with detected mutations in at least 10 tumors and 18 genes that were mutated in at least 2 tumors (78%) (FIG. 5). Interestingly, PAX3 was the most frequent mutated gene resulting in mRNA upregulation, while NRG1 was also altered at CNV level. Overall, while LMS02 and LMS04 were less altered, 85% of tumors were affected with at least eleven mutations, indicating the complexity of the tumorigenic process (FIG. 5).

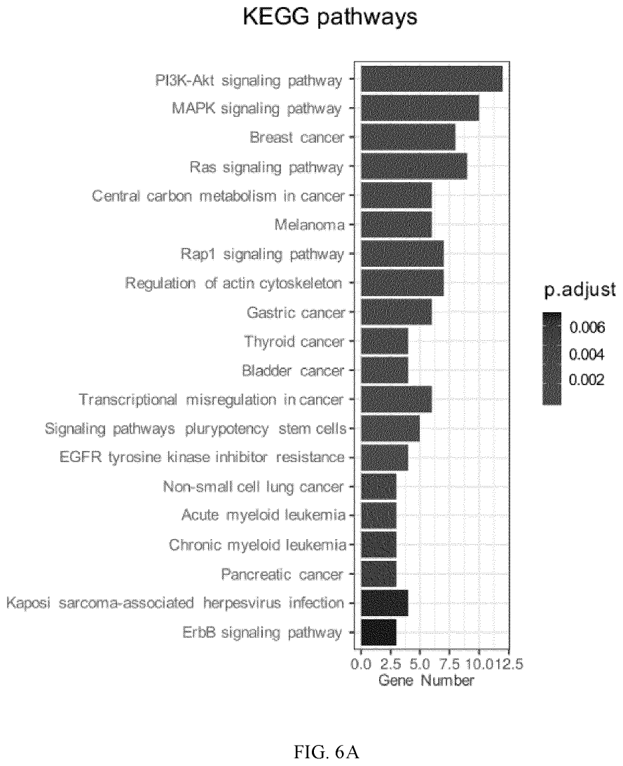

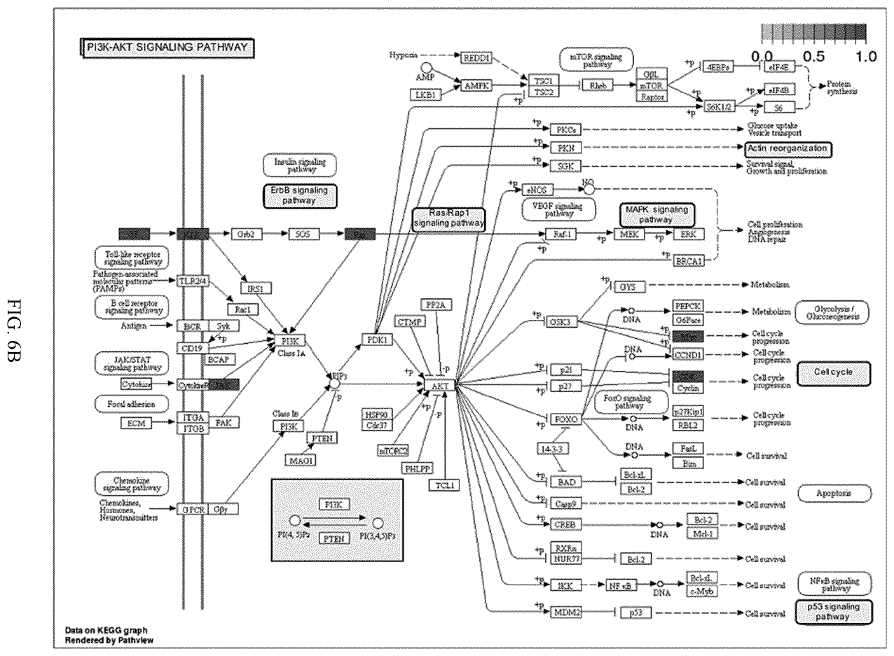

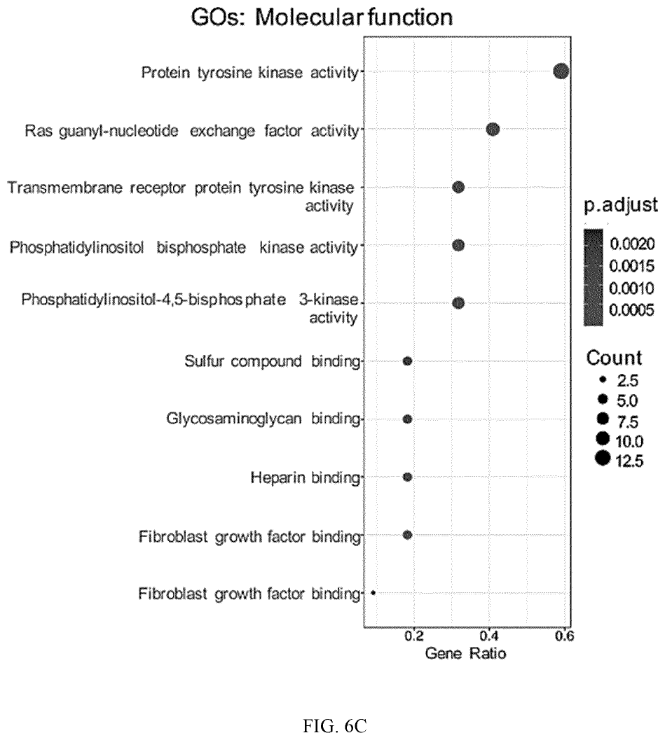

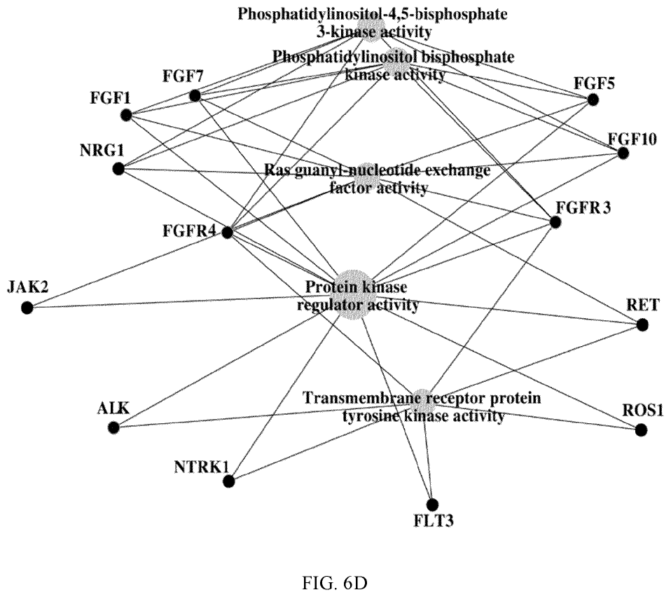

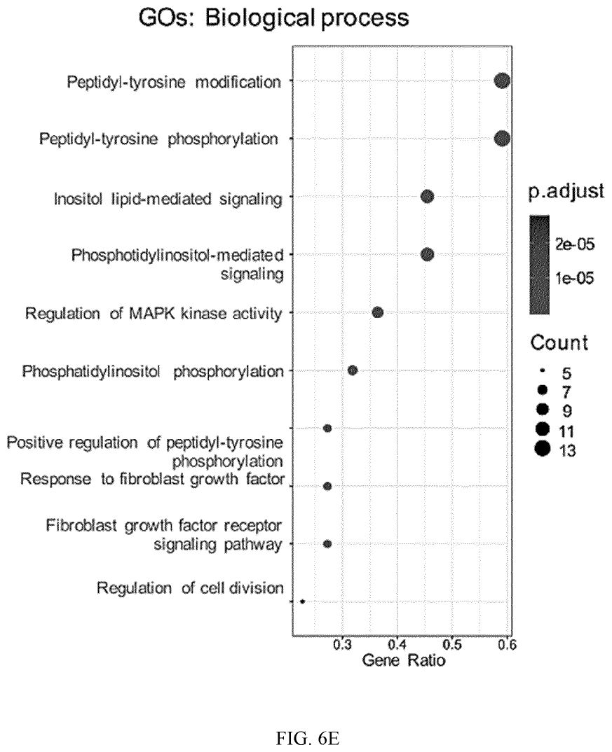

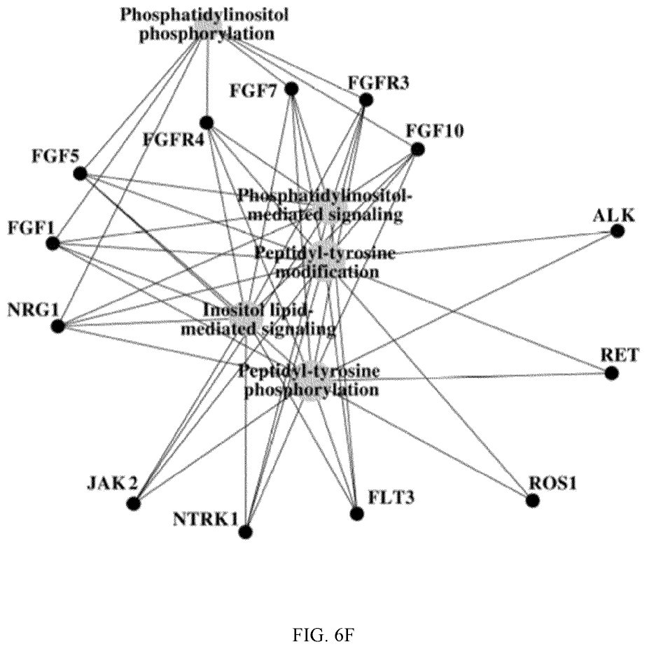

The KEGG database identified 20 pathways, mainly related to cancer and cell cycle, being the PI3K/AKT pathway the most representative (FIG. 6A). Main upregulated genes were identified from the integrative analysis as well as interactions with other represented pathways, such as RAS/RAP1 signaling pathway, MAPK, and p53 (FIG. 6B).

Implicated molecular functions (FIGS. 6C, 6D) and biological processes (FIGS. 6E, 6F) for LMS established a relationship network with interesting connections. The molecular function network highlighted 5 significant categories: protein-tyrosine kinase activity, ras guanyl-nucleotide exchange factor activity, transmembrane receptor protein tyrosine kinase activity, phosphatidylinositol biphosphate 3-kinase activity and phosphatidylinositol-4-5-biphosphate 3-kinase activity (FIG. 6C). All functions were connected by integrated genes that belonged to more than one function. Specifically, genes from the FGF family were shared by all functions (FIG. 6D). Regarding biological processes, peptidyl-tyrosine modification and phosphorylation as well as inositol lipid/phosphate-mediated signaling were the most representative processes (FIG. 6E), regulated by ALK, FLT3, ROS1, RET, NTRK1, JAK2, and FGF family genes, the latter with more shared functions than observed for molecular function (FIG. 6F).

### Remarks on Examples 1-5

Principal Findings

The present disclosure offers an innovative tool that allows clinicans to utilize genomic tools, genetic variants and possible transcriptomic and genomic markers in a new tool to effectuate the differential molecular diagnosis of myometrial tumors/uterine neoplasms such as LM, LMS and IMT. This provides a solution to a major problem in the current clinical approach to common uterine neoplasms by providing a tool that clinicals can use to evaluate the risk that apparently benign tumors are in fact rarer but much more dangerous malignant neoplasms. Based on the databases developed by the inventors, it is proposed that a diagnostic tool driven principally by “Next Generation Sequencing” of DNA and RNA originating in the neoplastic tissue differentiates uterine LMS and LM is a manner that cannot be achieved by histological techniques or any other current diagnostic method.

Results in Context

Numerous genes can influence tumor progression via several types of variations.⁴²⁻⁴⁸ The findings indicate that LMS are more unstable, with higher incidence and heterogenicity than LM. Specifically, most cases analyzed for CNVs demonstrated more losses than gains, being also present in some chromosomal regions that contain fibroblast growth gene (FGF1), proto-oncogenes like KRAS and non-receptor tyrosine kinase genes such as JAK2. Additionally, 29 exclusive affected genes in LMS were found, while only 4 were present in LM.

PCA allowed the gain of an overview of the data, showing that samples with the same tumor type clustered together with only two outliers: LMS12 and IMT01 (initially diagnosed as LMS01). These results were then confirmed with an associated dendrogram which was divided into two main branches, one containing tight clusters of LM (cluster1), and another with a majority of LMS (cluster2). To note, LMS08 and LMS13 had an intermediate pattern between LM and LMS, suggesting an additional molecular subtype. However, since they were commercially obtained, there are some limitations to validate the results as well as to obtain their clinical profile. Conversely, it was confirmed that IMT01 represented an additional molecular subtype,

At the transcriptomic level, 3 groups were identified: a homogeneous group with LMS samples (cluster1), a homogeneous group composed by LM (cluster2) and lastly a heterogeneous group composed by LM, LMS and IMT specimens (cluster3), which were confirmed by hierarchical clustering and gene set enrichment. Additionally, differential expression of PAX3, PAX7, ROS1, and TMPRSS2 may contribute to classify the outliers. Deep analysis in samples with an intermediate pattern is important and should be considered as a putative warning for further clinical analysis. In that sense, GO and gene set enrichment analysis provide structured functional and biological process information about these individual genes, since pathways involved in transcriptional misregulation and central carbon metabolism in cancer were overrepresented. Additionally, key pathways of RAS/MAPK and PI3K-AKT, which play important roles in cancer-related processes, such as cell growth, survival, and apoptosis, were also identified. These results agree with earlier published studies.⁴⁹

Furthermore, a novel TNS1-ALK fusion was identified in IMT01 sample, initially diagnosed as LMS. TNS1 encodes tensin 1, which crosslinks actin filaments and acts as an oncogenic driver in chromosomally unstable colorectal cancer.^{33,50} ALK is frequently found in fusions in patients with non-small cell lung cancer⁵¹⁻⁵³ as well as in inflammatory myofibroblastic tumors (IMT) of the female genital tract.⁵⁴ Since these are under-recognized smooth muscle tumors, the distinction between IMT, LM, and LMS can be subtle.⁵⁵ In fact, IMT01 sample, previously diagnosed as LMS, was instead established as IMT when ALK staining was detected by IHC and confirmed the translocation by FISH.

The integrated analysis also revealed numerous potential target genes like FGFR4, PAX3, PAX7, ROS1 and TMPRSS2 with detected mutations in at least 10 tumors. Among them, PAX3 was the most frequent mutated gene resulting in mRNA upregulation, while NRG1 was also altered at CNV level. Interestingly, it has been reported that, dysregulation of PAX family members, contributes to tumorigenesis in soft tissue sarcomas by altering signaling pathways that affect proliferation, cell death, myogenic differentiation, and migration. ⁵⁶Regarding, NRG1, there is evidence that acts as tumor suppressor gene and its dysregulation has been linked to tumorigenesis. ⁵⁷⁻⁵⁸

To better understand the tumorigenic process, networks between functions and genes, highlighted protein tyrosine kinase activity and peptidyl-tyrosine phosphorylation as the main categories for molecular functions and biological process respectively. Interestingly, both receptor and non-receptor tyrosine kinases have emerged as clinically useful drug target molecules for treating certain types of cancer, being LMS highly expressed tyrosine kinase another potential drug targetable cancer.

Clinical Implications

The main problem in diagnosing LM and LMS is the absence of risk factors and standardized criteria to identify them prior to surgery as benign or malignant, since currently there are no molecular biomarkers utilized in clinical practice. This situation could be the origin of significant stress in the patient, leading to unnecessary invasive procedures, and additional costs to the National System of Health.

Nowadays, the application of NGS enables the detection of new mutations that, when coupled to bioinformatic tools, advances the understanding of chromosomal/genetic instability.

The translational application of this reported differential panel is being explored in circulating cell-free tumor DNA (cftDNA) using tissue and/or liquid biopsy. Detection of cftDNA is now a reality as a biomarker for the detection of tumor DNA mutations in peripheral blood, urine, or other fluids as personalized therapy in cancer diagnosis as well as tumor progression⁵⁹ and the gynecological cancers should be no exception.

Some reports have described the use of NGS in LM and LMS,^{27-29,32} suggesting differing mechanisms underlie these tumorigenic mutations. In this sense, the study demonstrates consistent genetic differences between LM and LMS.

At the RNA level, recent studies have highlighted the importance of gene fusions and splice variants in solid tumors, because a single chimeric RNA transcript could result from numerous DNA alterations.^{60,61} A novel TNS1-ALK fusion was identified in one IMT sample, previously diagnosed as LMS. Fortunately, IMT has a less aggressive clinical course compared to most metastatic LMS, as was the case of the patient analyzed in the study, who remained alive with disease after two years (Table 6). In this sense, molecular diagnosis could overcome the limitations of conventional analyses.

Finally, pathways differentially affected in LM versus LMS have been identified. In fact, the comparison of the results with previously published studies reinforces the importance of certain specific pathways such as RAS/RAP1 signaling pathway, MAPK, and p53.^{62,63} For instance, the PI3K/AKT/mTOR pathway is activated in ˜30%-40% of breast cancer cases. In triple-negative breast cancer, oncogenic activation of the PI3K/AKT/mTOR pathway can happen as a function of overexpression of upstream regulators such as EGFR, activating mutations of PIK3CA, loss of function or expression of PTEN, and the proline-rich inositol polyphosphatase, which are downregulators of PI3K. This is consistent with the hypothesis that PI3K inhibitors can overcome resistance to endocrine therapy.

### REFERENCES

The following references are cited within the present Application. Each is incorporate herein by reference in their entireties.

1. Baird D D, Dunson D B, Hill M C, Cousins D, Schectman J M. High cumulative incidence of uterine leiomyoma in black and white women: ultrasound evidence. Am J Obstet Gynecol 2003; 188:100-7.

2. Parker W H. Etiology, symptomatology, and diagnosis of uterine myomas. Fertil Steril 2007; 87:725-36.

3. Bulun S E. Uterine fibroids. N Engl J Med. 2013; 369:1344.

4. Donnez J, Tatarchuk T F, Bouchard P, et al. Ulipristal acetate versus placebo for fibroid treatment before surgery. N Engl J Med. 2012; 366:409-20.

5. Donnez J, Dolmans M M. Uterine fibroid management: from the present to the future. Hum Reprod Update 2016; 22:665-86.

6. AAGL practice report: Morcellation during uterine tissue extraction. AAGL Advancing Minimally Invasive Gynecology Worldwide. J Minim Invasive Gynecol 2014; 21:517-30.

7. Bhave Chittawar P, Franik S, Pouwer A W, Farquhar C. Minimally invasive surgical techniques versus open myomectomy for uterine fibroids. Cochrane Database Syst Rev 2014; 10:CD004638.

8. Bogani G, Ditto A, Martinelli F, et al. Morcellator's port-site metastasis of a uterine smooth muscle tumor of uncertain malignant potential after minimally invasive myomectomy. J Minim Invasive Gynecol 2016; 23:647-9.

9. Skorstad M, Kent A, Lieng M. Uterine leiomyosarcoma-13 incidence, treatment, and the impact of morcellation. A nationwide cohort study. Acta Obstet Gynecol Scand 2016; 95:984-90.

10. Giuntoli RL I I, Metzinger D S, DiMarco C S, et al. Retrospective review of 208 patients with leiomyosarcoma of the uterus: prognostic indicators, surgical management, and adjuvant therapy. Gynecol Oncol 2003; 89:460-9.

11. Kobayashi H, Uekuri C, Akasaka J, et al. The biology of uterine sarcomas: A review and update. Mol Clin Oncol 2013; 1:599-609.

12. Lusby K, Savannah K B, Demicco E G, et al. Uterine leiomyosarcoma management, outcome, and associated molecular biomarkers: a single institution's experience. Ann Surg Oncol 2013; 20:2364-72.

13. Amant F, Coosemans A, Debiec-Rychter M, Timmerman D, Vergote I. Clinical management of uterine sarcomas. Lancet Oncol 2009; 10:1188-98.

14. Brown J, Taylor K, Ramirez P T, et al. Laparoscopic supracervical hysterectomy with morcellation: should it stay or should it go? J Minim Invasive Gynecol 2015; 22:185-92.

15. Kho K A, Nezhat C H. Evaluating the risks of electric uterine morcellation. JAMA 2014; 311:905-6.

16. Pritts E A, Parker W H, Brown J, Olive D L. Outcome of occult uterine leiomyosarcoma after surgery for presumed uterine fibroids: a systematic review. J Minim Invasive Gynecol 2015; 22:26-33.

17. Parker W, Berek J S, Pritts E, et al. An open letter to the Food and Drug Administration regarding the use of morcellation procedures in women having surgery for presumed uterine myomas. J Minim Invasive Gynecol 2016; 23:303-8.

18. Halaska M J, Haidopoulos D, Guyon F, et al. European Society of Gynecological Oncology statement on fibroid and uterine morcellation. Int J Gynecol Cancer 2017; 27:189-92.

19. Levitz J. Fibroid surgery puts doctor fighting cancer diagnosis in the spotlight. Wall Street J 2013, December. Retrieved from: http://online.wsj.com/news/articles/SB10001424052702304866904579266714199128946 [2013, Sep. 15].

20. Roehr B. Amy Josephine Reed. BMJ 2017; 357: j2827.

21. Rousseau M, Morel A, Dechoux S, et al. Can the risks associated with uterine sarcoma morcellation really be prevented? Overview of the role of uterine morcellation in 2018. J Gynecol Obstet Hum Reprod 2018; 47(8):341-349.

22. Brölmann H, Tanos V, Grimbizis G, et al. European Society of Gynaecological Endoscopy (ESGE) steering committee on fibroid morcellation. Options on fibroid morcellation: a literature review. Gynecol Surg 2015; 12:3-15.

23. Shwayder J, Sakhel K. Imaging for uterine myomas and adenomyosis. J Minim Invasive Gynecol 2014; 21:362-76.

24. Cho H Y, Kim K, Kim Y B, No J H. Differential diagnosis between uterine sarcoma and leiomyoma using preoperative clinical characteristics. J Obstet Gynaecol Res 2016; 42:313-8.

25. Shah S H, Jagannathan J P, Krajewski K, O'Regan K N, George S, Ramaiya NH. Uterine sarcomas: then and now. AJR Am J Roentgenol 2012; 199:213-23.

26. Leiser A L, Anderson S E, Nonaka D, et al. Apoptotic and cell cycle regulatory markers in uterine leiomyosarcoma. Gynecol Oncol 2006; 101:86-91.

27. Ravegnini G, Mariño-Enriquez A, Slater J, et al. MED12 mutations in leiomyosarcoma and extrauterine leiomyoma. Mod Pathol 2013; 26:743-9.

28. Bertsch E, Qiang W, Zhang Q, et al. MED12 and HMGA2 mutations: two independent genetic events in uterine leiomyoma and leiomyosarcoma. Mod Pathol 2014; 27:1144-53.

29. Jour G, Scarborough J D, Jones R L, et al. Molecular profiling of soft tissue sarcomas using next-generation sequencing: a pilot study toward precision therapeutics. Hum Pathol 2014; 45:1563-71.

30. Mehine M, Mäkinen N, Heinonen H R, Aaltonen L A, Vahteristo P. Genomics of uterine leiomyomas: insights from high-throughput sequencing. Fertil Steril 2014; 102(3):621-9.

31. Mäkinen N, Kämpjärvi K, Frizzell N, Bützow R, Vahteristo P. Characterization of MED12, HMGA2, and FH alterations reveals molecular variability in uterine smooth muscle tumors. Mol Cancer 2017; 16(1):101.

32. Mäkinen N, Aavikko M, Heikkinen T, et al. Exome sequencing of uterine leiomyosarcomas identifies frequent mutations in TP53, ATRX, and MED12. PLoS Genet 2016; 12:e1005850.

33. Cuppens T, Moisse M, Depreeuw J, et al. Integrated genome analysis of uterine leiomyosarcoma to identify novel driver genes and targetable pathways. Int J Cancer 2018; 142(6):1230-43.

34. Amant F, Van den Bosch T, Vergote I, Timmerman D. Morcellation of uterine leiomyomas: a plea for patient triage. Lancet Oncol. 2015; 16(15):1454-56.

35. Hendrickson M R, Tavassoli F A, Kempson R L, McCluggage W G, Haller U, Kubik-Huch RA 2003. Mesenchymal tumours and related lesions In: Tavassoli F A, Devilee P, eds. Tumours of the Breast and Female Genital Organs. Lyon, France: IARC; pp. 233.

36. Fletcher C D. The evolving classification of soft tissue tumours—an update based on the new 2013 WHO classification. Histopathology 2014; 64:2-11.

37. Dunn T, Berry G, Emig-Agius D, et al. Pisces: an accurate and versatile variant caller for somatic and germline next-generation sequencing data. Bioinformatics 2018 [Epub ahead of print].

38. Robinson M D, McCarthy D J, Smyth G K. edgeR: a Bioconductor package for differential expression analysis of digital gene expression data. Bioinformatics. 2010; 26:139-40.

39. Gentleman R C, Carey V J, Bates D M, et al. Bioconductor: open software development for computational biology and bioinformatics. Genome Biol 2004; 5(10):R80.

40. Yatabe Y. ALK FISH and IHC: you cannot have one without the other. J Thorac Oncol 2015; 10:548-50.

41. Savic S, Diebold J, Zimmermann A K, et al. Screening for ALK in non-small cell lung carcinomas: 5A4 and D5F3 antibodies perform equally well, but combined use with FISH is recommended. Lung Cancer 2015; 89(2):104-9.

42. Sandberg A A. Updates on the cytogenetics and molecular genetics of bone and soft tissue tumors: leiomyosarcoma. Cancer Genet Cytogenet 2005; 161(1):1-19.

43. Yang J, Du X, Chen K, et al. Genetic aberrations in soft tissue leiomyosarcoma. Cancer Lett 2009; 275(1):1-8.

44. Raish M, Khurshid M, Ansari M A, et al. Analysis of molecular cytogenetic alterations in uterine leiomyosarcoma by array-based comparative genomic hybridization. J Cancer Res Clin Oncol 2012; 138(7):1173 -86.

45. Lehtonen R, Kiuru M, Vanharanta S et al. Biallelic inactivation of fumarate hydratase (FH) occurs in nonsyndromic uterine leiomyomas but is rare in other tumors. Am J Pathol. 2004; 164(1): 17-22.

46. Pérot G, Croce S, Ribeiro A, et al. MED 12 alterations in both human benign and malignant uterine soft tissue tumors. PLoS One. 2012; 7(6):e40015.

47. Mehine M, Kaasinen E, Mäkinen N et al. Characterization of uterine leiomyomas by whole-genome sequencing. N Engl J Med. 2013; 369(1):43-53.

48. Slatter T L, Hsia H, Samaranayaka A. Loss of ATRX and DAXX expression identifies poor prognosis for smooth muscle tumours of uncertain malignant potential and early stage uterine leiomyosarcoma. J Pathol Clin Res. 2015 Mar. 16 ; 1(2):95-105.

49. Cuppens T, Tuyaerts S, Amant F. Potential therapeutic targets in uterine sarcomas. Sarcoma 2015; 2015:243298.

50. Burghel G J, Lin W Y, Whitehouse H, et al. Identification of candidate driver genes in common focal chromosomal aberrations of microsatellite stable colorectal cancer. PLoS One 2013; 8:e83859.

51. Caliò A, Nottegar A, Gilioli E, et al. ALK/EML4 fusion gene may be found in pure squamous carcinoma of the lung. J Thorac Oncol 2014; 9(5):729-32.

52. Chapman A M, Sun K Y, Ruestow P, Lung cancer mutation profile of EGFR, ALK, and KRAS: Meta-analysis and comparison of never and ever smokers. Lung Cancer. 2016; 102:122-134.

53. Dubey A P, Pathi N, Viswanath S. New insights into anaplastic lymphoma kinase-positive nonsmall cell lung cancer. Indian J Cancer. 2017; 54(1):203-208.

54. Pickett J L, Chou A, Andrici J A, et al. Inflammatory myofibroblastic tumors of the female genital tract are under-recognized: a low threshold for ALK immunohistochemistry is required. Am J Surg Pathol 2017; 41(10):1433-42.

55. Parra-Herran C, Schoolmeester J K, Yuan L, et al. Myxoid leiomyosarcoma of the uterus: a clinicopathologic analysis of 30 cases and review of the literature with reappraisal of its distinction from other uterine myxoid mesenchymal neoplasms. Am J Surg Pathol 2016; 40(3):285-301.

56. Wang Q, Fang W H, Krupinski J, Kumar S, Slevin M, Kumar P. “Pax genes in embryogenesis and oncogenesis”. review. Journal of Cellular and Molecular Medicine 2008; 12(6A): 2281-94.

57. Yatsenko S A, Mittal P, Wood-Trageser M A, et al. Highly heterogeneous genomic landscape of uterine leiomyomas by whole exome sequencing and genome-wide arrays. Fertil Steril. 2017; 107(2):457-466. e9.

58. Chua Y L, Ito Y, Pole J C, et al. The NRG1 gene is frequently silenced by methylation in breast cancers and is a strong candidate for the 8p tumour suppressor gene. Oncogene 2009; 28(46): 4041-52.

59. Bettegowda C, Sausen M, Leary R J, et al. Detection of circulating tumor DNA in early- and late-stage human malignancies. Sci Transl Med 2014; 6(224):224ra224.

60. Maher C A, Kumar-Sinha C, Cao X, et al. Transcriptome sequencing to detect gene fusions in cancer. Nature 2009; 458(7234):97-101.

61. Klijn C, Durinck S, Stawiski E W, et al. A comprehensive transcriptional portrait of human cancer cell lines. Nat Biotechnol 2015; 33(3):306-12.

62. Raphael J, Desautels D, Pritchard K I, Petkova E, Shah P S. Phosphoinositide 3-kinase inhibitors in advanced breast cancer: A systematic review and meta-analysis. Eur J Cancer 2018; 91:38-46.

63. Costa R L B, Han H S, Gradishar W J. Targeting the PI3K/AKT/mTOR pathway in triple-negative breast cancer: a review. Breast Cancer Res Treat 2018; 169(3):397-406.

64. Lek M, Karczewski K J, Minikel E V, et al. Analysis of protein-coding genetic variation in 60,706 humans. Nature 2016; 536:285-91.

65. Yu G, Wang L G, Han Y, He Q Y. clusterProfiler: an R package for comparing biological themes among gene clusters. OMICS 2012; 16(5):284-7.

66. Houang M, Toon C W, Clarkson A, et al. Reflex ALK immunohistochemistry is feasible and highly specific for ALK gene rearrangements in lung cancer. Pathology 2014; 46:383-8.

67. Kanehisa M, Goto S, Sato Y, Furumichi M, Tanabe M. KEGG for integration and interpretation of large-scale molecular data sets. Nucleic Acids Res 2012; 40:D109-14.

68. Wickham H. ggplot2: Elegant Graphics for Data Analysis. Springer-Verlag New York, 2016.

69. Wickham H. tidyverse: Easily Install and Load the ‘Tidyverse’. R package version 1.2.1. https://CRAN.R-project.org/package=tidyverse. 2017.

70. Chen H. VennDiagram: Generate High-Resolution Venn and Euler Plots. R package version 1.6.17. https://CRAN.R-project.org/package=VennDiagram. 2016.

71. Obenchain V, Lawrence M, Carey V, Gogarten S, Shannon P, Morgan M. VariantAnnotation: a Bioconductor package for exploration and annotation of genetic variants. Bioinformatics 2014; 30:2076-78.

72. Zhao S, Guo Y, Sheng Q, Shyr Y. Advanced heat map and clustering analysis using heatmap3. Biomed Res Int 2014; 2014:986048. R package version 1.1.1. https://CRAN.R-project.org/package=heatmap3.

73. Huber W, Carey V J, Gentleman R, et al. Orchestrating high-throughput genomic analysis with Bioconductor. Nat Methods 2015; 12:115-21.

1-63. (canceled)

64. A method of diagnosing whether a myometrial tumor comprises uterine leiomyosarcoma comprising detecting one or more biomarkers indicative of a uterine leiomyosarcoma genotype in a sample from the subject and wherein the one or more biomarkers indicative of a uterine leiomyosarcoma genotype comprise:
(i) an upregulation in the mRNA of one or more of the following genes: BRCA2, ALK, FGFR3, FGFR4, FLT3, NTRK1, PAX3, PAX7, RET, ROS1, or TMPRSS2 gene;
(ii) a copy number variant (CNV) duplication mutation in one or more of the following biomarkers: CDK4, FGF10, FGF5, MYC, MYCL1, or NRG I;
(iii) a CNV deletion mutation in one or more of the following biomarkers: FGF1, FGF14, JAK2, or KRAS;
(iv) a CNV deletion and duplication mutation in one or more of the following biomarkers: FGF14, FGF7, MDM4, MYCL1, or NRG 1; or
(v) a single nucleotide variant (SNV) mutation in one or more of the following biomarkers: FGF5 and RET.

65. The method according to claim 64, wherein the genotype indicative of leiomyosarcoma is obtained by a genotyping assay on a biological sample of the subject or by detecting transcript levels on a biological sample of the subject.

66. The method of claim 64, wherein the biological sample is a biopsy of the myometrial tumor.

67. The method of claim 64 further comprising obtaining a DNA sample or an RNA sample from the biological sample.

68. A method of diagnosing whether a myometrial tumor comprises uterine leiomyoma comprising detecting one or more biomarkers indicative of a uterine leiomyoma genotype in a sample from the subject and wherein the one or more biomarkers indicative of a uterine leiomyoma genotype comprises
(i) a copy number variant (CNV) duplication mutation in the CCND1 or in the FGFR3 gene or
(ii) a CNV deletion mutation in the MET gene.

69. A method for treating a myometrial tumor in a subject comprising: (a) confirming with a genotyping assay that the tumor does not contain a uterine leiomyosarcoma genotype and (b) surgically removing the myometrial tumor if it is confirmed that the subject does not have a uterine leiomyosarcoma genotype, wherein the uterine leiomyosarcoma genotype comprises:
(i) an upregulation in the mRNA of one or more of the following genes: BRCA2, ALK, FGFR3, FGFR4, FLT3, NTRK1, PAX3, PAX7, RET, ROS1, or TMPRSS2 gene;
(ii) a copy number variant (CNV) duplication mutation in one or more of the following biomarkers: CDK4, FGF10, FGF5, MYC, MYCL1, and NRG I;
(iii) a CNV deletion mutation in one or more of the following biomarkers: FGF1, FGF14, JAK2, and KRAS;
(iv) a CNV deletion and duplication mutation in one or more of the following biomarkers: FGF14, FGF7, MDM4, MYCL1, and NRG 1; or
(v) a SNV mutation in one or more of the following biomarkers: FGF5 and RET.

70. The method of claim 69, wherein the myometrial tumor is surgically removed if it is further confirmed that the subject has a uterine leiomyoma genotype comprising:
a CNV duplication mutation in the CCND or in the FGFR3 gene; or
(ii) a CNV deletion mutation in MET.

71. The method of claim 69, wherein the step of surgical removal of the myometrial tumor is by myomectomy.

72. The method of claim 71, wherein the myomectomy is carried out by laparoscopic morcellation.

73. The method of claim 70, wherein it is confirmed that the subject has a uterine leiomyoma genotype.

74. The method of claim 73, wherein the uterine leiomyoma is a subserous fibroid, an intramural fibroid, or a submucous fibroid.

75. The method of claim 73, wherein the uterine leiomyoma is a submucous leiomyoma having a grade 0, grade 1, or grade 2 uterine leiomyoma.

76. The method according to claim 69, wherein the genotype indicative of leiomyosarcoma is obtained by a genotyping assay on a biological sample of the subject or by detecting transcript levels on a biological sample of the subject.

77. The method of claim 76, wherein the biological sample is a biopsy of the myometrial tumor.

78. The method of claim 76, further comprising obtaining a DNA sample or an RNA sample from the biological sample.

79. The method of claim 76, wherein the genotyping assay is a restriction fragment length polymorphism identification (RFLPI) of the DNA sample, a random amplified polymorphic detection (RAPD) of the DNA sample, an amplified fragment length polymorphism (AFLPD) of the DNA sample, a polymerase chain reaction (PCR) of the DNA sample, DNA sequencing of the DNA sample, hybridization of the DNA sample to a nucleic acid microarray or by next generation sequencing.

80. The method according to claim 76, wherein the detection of transcript levels from the RNA sample is carried out by method selected from serial analysis of gene expression (SAGE), cap analysis of gene expression (CAGE), and massively parallel signature sequencing (MPSS), nanopore sequencing, sequencing by ligation (SOLid), combinatorial probe anchor synthesis, pyrosequencing, ion torrent sequencing, sequencing by synthesis or next generation sequencing.

81. The method of claim 80, wherein the next-generation sequencing method is single-molecule real-time sequencing (SMRT), ion semiconductor sequencing, pyrosequencing, sequencing by synthesis, combinatorial probe anchor synthesis (cPAS), sequencing by ligation (SOLiD sequencing), nanopore sequencing, or massively parallel signature sequencing (MPSS).

82. The method of claim 68, further comprising performing a genotyping assay on a biological sample from the subject to determine whether the subject has a uterine leiomyosarcoma genotype.

83. A method of treating a myometrial tumor in a subject comprising, (a) confirming with a genotyping assay that a subject has a uterine leiomyosarcoma genotype; and (b) surgically removing the myometrial tumor by hysterectomy if the subject is found to have uterine leiomyosarcoma genotype, wherein the uterine leiomyosarcoma genotype comprises:
(i) an upregulation in the mRNA of one or more of the following genes: BRCA2, ALK, FGFR3, FGFR4, FLT3, NTRK1, PAX3, PAX7, RET, ROS1, or TMPRSS2 gene,
(ii) a copy number variant (CNV) duplication mutation in one or more of the following biomarkers: CDK4, FGF10, FGF5, MYC, MYCL1, and NRG I,
(iii) a CNV deletion mutation in one or more of the following biomarkers: FGF1, FGF14, JAK2, and KRAS,
(iv) a CNV deletion and duplication mutation in one or more of the following biomarkers: FGF14, FGF7, MDM4, MYCL1, and NRG 1 or
(v) a single nucleotide variant (SNV) mutation in one or more of the following biomarkers: FGF5 and RET, wherein the one or more mutations are indicative of a uterine leiomyosarcoma genotype.
